SEQUENCE LISTING (136 sequences)

SEQ ID NO: 1 - protein, 5 aa, Alpaca
KLGMG

SEQ ID NO: 2 - protein, 17 aa, Alpaca
VISASGLNTWYGDSVKG

SEQ ID NO: 3 - protein, 13 aa, Alpaca
GPRLRVQDPQYDY

SEQ ID NO: 4 - protein, 122 aa, Alpaca
QVQLVESGGGLVQAGGSLRLSCVASERTFSKLGMGWFRQAPGKEREFVSVISASGLNTWYGDSVKGRFTISRDTATNTIYLQMNSLKPEDTAVYYCAAGPRLRVQDPQYDYWGQGTQVTVSS

SEQ ID NO: 5 - protein, 5 aa, Alpaca
KLGMG

SEQ ID NO: 6 - protein, 17 aa, Alpaca
VISASGLNTWTGDSVKG

SEQ ID NO: 7 - protein, 13 aa, Alpaca
GPRLRVQDPQYDY

SEQ ID NO: 8 - protein, 122 aa, Alpaca
QVQLVESGGGLVQAGGSLRLSCVGSERTFSKLGMGWFRQAPGKEREFVSVISASGLNTWTGDSVKGRFTISRDTATNTVYLQMNSLKPEDTAVYYCAAGPRLRVQDPQYDYWGQGTQVTVSS

SEQ ID NO: 9 - protein, 5 aa, Alpaca
KLGMG

SEQ ID NO: 10 - protein, 17 aa, Alpaca
VISASGLNTWYGDSVKG

SEQ ID NO: 11 - protein, 13 aa, Alpaca
GPRLRVQDPQYDY

SEQ ID NO: 12 - protein, 122 aa, Alpaca
QVQLVESGGGLVQAGGSLRLSCVGSERTFNKLGMGWFRQAPGKEREFVSVISASGLNTWYGDSVKGRFTISRDTATNTIYLQMNSLKPEDTAVYYCAAGPRLRVQDPQYDYWGQGTQVTVSS

SEQ ID NO: 13 - protein, 5 aa, Alpaca
KFGAG

SEQ ID NO: 14 - protein, 17 aa, Alpaca
GLTAIGGTAIYADSVKG

SEQ ID NO: 15 - protein, 13 aa, Alpaca
GVRQDLRVADYYY

SEQ ID NO: 16 - protein, 121 aa, Alpaca
QVQLVESGGGLVQAGGSLRLSCAASGRSIRKFGAGWFRQAPGKEREFVAGLTAIGGTAIYADSVKGRFTISRDNAKNTASLVMNSLRPEDTAVYYCAAGVRQDLRVADYYYWGQGTQVTVS

SEQ ID NO: 17 - protein, 5 aa, Alpaca
KLGMG

SEQ ID NO: 18 - protein, 17 aa, Alpaca
VISASGLNTWYGDSVKG

SEQ ID NO: 19 - protein, 13 aa, Alpaca
GPRLRVQDPQHDY

SEQ ID NO: 20 - protein, 122 aa, Alpaca
QVQLVESGGGLVQAGGSLRLSCVASERTFSKLGMGWFRQAPGKEREFVSVISASGLNTWYGDSVKGRFTISRDTATNTVYLQMNSLKPEDTAVYYCAAGPRLRVQDPQHDYWGQGTQDTVSS

SEQ ID NO: 21 - protein, 5 aa, Alpaca
KLGMG

SEQ ID NO: 22 - protein, 17 aa, Alpaca
VISATGLNTWYGDSVKG

SEQ ID NO: 23 - protein, 13 aa, Alpaca
GPRLRVQHPHYDY

SEQ ID NO: 24 - protein, 122 aa, Alpaca
QVQLVESGGGLVQAGGSLRLSCVASERTFNKLGMGWFRQAPGKEREFVSVISATGLNTWYGDSVKGRFTISRDTATNTVYLQMNSLKPEDTAVYYCAVGPRLRVQHPHYDYWGQGTQVTVSS

SEQ ID NO: 25 - protein, 5 aa, Alpaca
PHAIG

SEQ ID NO: 26 - protein, 17 aa, Alpaca
CISSSGYNTYYAEPVEG

SEQ ID NO: 27 - protein, 16 aa, Alpaca
VLYGGLAGCEAIGTDY

SEQ ID NO: 28 - protein, 125 aa, Alpaca
QVQLVESGGGLVQPGGSLRLSCAESGAVVKPHAIGWFRQVPGKERERVGCISSSGYNTYYAEPVEGRFTISRDNAKNTVYLQMNSLKPEDTAVYYCALVLYGGLAGCEAIGTDYWGKGTLVTVSS

SEQ ID NO: 29 - protein, 5 aa, Alpaca
RYAMA

SEQ ID NO: 30 - protein, 17 aa, Alpaca
SIGGSGVSTNYADSVKG

SEQ ID NO: 31 - protein, 19 aa, Alpaca
GTNFYKGLNYYTGVRNYDS

SEQ ID NO: 32 - protein, 128 aa, Alpaca
QVQLVESGGGLVQAGGSLRLSCAASGRTFSRYAMAWFRQAPGKEREFVASIGGSGVSTNYADSVKGRFTISRDNAKNTVTLQMMSLKPEDTGVYYCAAGTNFYKGLNYYTGVRNYDSWGQGTQVSVSS

SEQ ID NO: 33 - protein, 5 aa, Alpaca
FKSMG

SEQ ID NO: 34 - protein, 16 aa, Alpaca
AISSKGIMTNSDSVKG

SEQ ID NO: 35 - protein, 12 aa, Alpaca
VEVGVGGLVNVY

SEQ ID NO: 36 - protein, 120 aa, Alpaca
QVQLVESGGGLAQVGGSLRLSCAASGSTFSFKSMGWYREAPGNQRELVAAISSKGIMTNSDSVKGRFTISRDNAKNTVDLHLNNLKPEDTAVYYCNVVEVGVGGLVNVYWGQGTQVTVSS

SEQ ID NO: 37 - protein, 5 aa, Alpaca
KLGMG

SEQ ID NO: 38 - protein, 17 aa, Alpaca
VISASGLNTWYGDSVKG

SEQ ID NO: 39 - protein, 13 aa, Alpaca
GPRLRVQDPQYDY

SEQ ID NO: 40 - protein, 122 aa, Alpaca
QVQLVESGGGLVQAGGSLRLSCVASERTFNKLGMGWFRQAPGKEREFVSVISASGLNTWYGDSVKGRFTISRDTATNTIYLQMNSLKPEDTAVYYCAAGPRLRVQDPQYDYWGQGTQVTVSS

SEQ ID NO: 41 - protein, 5 aa, Alpaca
AYRMG

SEQ ID NO: 42 - protein, 17 aa, Alpaca
AISWNGDSTNYADSVKG

SEQ ID NO: 43 - protein, 13 aa, Alpaca
VKGIGVPPNMYDY

SEQ ID NO: 44 - protein, 122 aa, Alpaca
QVQLVESGGGLVQAGGSLRLSCAASGRTGNAYRMGWFRQAPGKEREFVAAISWNGDSTNYADSVKGRFTISRDNAKNTVYLQMNSLKPEDTAVYYCAVVKGIGVPPNMYDYWGQGTQVTVSS

SEQ ID NO: 45 - protein, 5 aa, Alpaca
INAMG

SEQ ID NO: 46 - protein, 16 aa, Alpaca
TISSGGSTYYADSVKG

SEQ ID NO: 47 - protein, 8 aa, Alpaca
LKGATPGY

SEQ ID NO: 48 - protein, 116 aa, Alpaca
QVQLVESGGGLVQAGGSLRLSCAASGSDFSINAMGWYRQAPGKERELVATISSGGSTYYADSVKGRFTISRDNAKNTLYLHMNTLKPEDTAMYYCAILKGATPGYWGQGTQVTVSS

SEQ ID NO: 49 - protein, 5 aa, Alpaca
VYAIG

SEQ ID NO: 50 - protein, 17 aa, Alpaca
CVSSSGDSTKYADSVKG

SEQ ID NO: 51 - protein, 14 aa, Alpaca
VHPSSCHTKPGFGS

SEQ ID NO: 52 - protein, 123 aa, Alpaca
QVQLVESGGGLVQPGGSLRLSCAASGFTLDVYAIGWFRQAPGKEREGVSCVSSSGDSTKYADSVKGRFTISRDNAKNTVYLQMNILKPEDTAVYYCAAVHPSSCHTKPGFGSWGPGTQVTVSS

SEQ ID NO: 53 - protein, 5 aa, Alpaca
SDGFG

SEQ ID NO: 54 - protein, 16 aa, Alpaca
DITSVGNTNYADSVKG

SEQ ID NO: 55 - protein, 11 aa, Alpaca
KKWRFGSWFDY

SEQ ID NO: 56 - protein, 119 aa, Alpaca
QVQLVESGGGLVQAGGSLRLSCAASGSIFSSDGFGWYGQAPGKQRELVADITSVGNTNYADSVKGRFTISRDNAKNTVYLQMNNLKPEDTAVYYCNTKKWRFGSWFDYWGQGTQVTVSS

SEQ ID NO: 57 - protein, 5 aa, Alpaca
VYAIG

SEQ ID NO: 58 - protein, 17 aa, Alpaca
CISSSGGSTNYADSVKG

SEQ ID NO: 59 - protein, 14 aa, Alpaca
VHPTSCHTKPGFDS

SEQ ID NO: 60 - protein, 123 aa, Alpaca
QVQLVESGGGLVQPGGSLRLSCAASGSTLDVYAIGWFRQAPGKEREGVSCISSSGGSTNYADSVKGRFTISRDNAKNTVYLQMNILKPEDTAVYHCAQVHPTSCHTKPGFDSWGPGTQVTVAS

SEQ ID NO: 61 - protein, 5 aa, Alpaca
INAIG

SEQ ID NO: 62 - protein, 16 aa, Alpaca
AISSGGSTNYADSVKG

SEQ ID NO: 63 - protein, 32 aa, Alpaca
RFTISRDNAKNTVYLQMNSLKPEDTAVYYCNI

SEQ ID NO: 64 - protein, 117 aa, Alpaca
QVQLVESGGGLVQAGGSLRLSCAASGSTFSINAIGWYRQAPGKQRELVAAISSGGSTNYADSVKGRFTISRDNAKNTVYLQMNSLKPEDTAVYYCNILKGGAAYNYWGQGTQVTVSS

SEQ ID NO: 65 - protein, 5 aa, Alpaca
KLGMG

SEQ ID NO: 66 - protein, 17 aa, Alpaca
VISASGLNTWYGDSVKG

SEQ ID NO: 67 - protein, 13 aa, Alpaca
GPRLRVQHPHYDY

SEQ ID NO: 68 - protein, 122 aa, Alpaca
QVQLVESGGGLVQAGGSLRLSCVASERTFSKLGMGWFRQAPGKEREFVSVISASGLNTWYGDSVKGRFTISRDTATNTVYLQMNSLKPEDTAVYYCAAGPRLRVQHPHYDYCGQGTQVTVSS

SEQ ID NO: 69 - protein, 8 aa, Alpaca
ERTFSKLG

SEQ ID NO: 70 - protein, 8 aa, Alpaca
ISASGLNT

SEQ ID NO: 71 - protein, 15 aa, Alpaca
AAGPRLRVQDPQYDY

SEQ ID NO: 72 - protein, 122 aa, Alpaca; identical to SEQ ID NO: 4

SEQ ID NO: 73 - protein, 8 aa, Alpaca
ERTFSKLG

SEQ ID NO: 74 - protein, 8 aa, Alpaca
ISASGLNT

SEQ ID NO: 75 - protein, 15 aa, Alpaca
AAGPRLRVQDPQYDY

SEQ ID NO: 76 - protein, 122 aa, Alpaca; identical to SEQ ID NO: 8

SEQ ID NO: 77 - protein, 8 aa, Alpaca
ERTFNKLG

SEQ ID NO: 78 - protein, 8 aa, Alpaca
ISASGLNT

SEQ ID NO: 79 - protein, 15 aa, Alpaca
AAGPRLRVQDPQYDY

SEQ ID NO: 80 - protein, 122 aa, Alpaca; identical to SEQ ID NO: 12

SEQ ID NO: 81 - protein, 8 aa, Alpaca
GRSIRKFG

SEQ ID NO: 82 - protein, 8 aa, Alpaca
LTAIGGTA

SEQ ID NO: 83 - protein, 15 aa, Alpaca
AAGVRQDLRVADYYY

SEQ ID NO: 84 - protein, 121 aa, Alpaca; identical to SEQ ID NO: 16

SEQ ID NO: 85 - protein, 8 aa, Alpaca
ERTFSKLG

SEQ ID NO: 86 - protein, 8 aa, Alpaca
ISASGLNT

SEQ ID NO: 87 - protein, 15 aa, Alpaca
AAGPRLRVQDPQHDY

SEQ ID NO: 88 - protein, 122 aa, Alpaca; identical to SEQ ID NO: 20

SEQ ID NO: 89 - protein, 8 aa, Alpaca
ERTFNKLG

SEQ ID NO: 90 - protein, 8 aa, Alpaca
ISATGLNT

SEQ ID NO: 91 - protein, 15 aa, Alpaca
AVGPRLRVQHPHYDY

SEQ ID NO: 92 - protein, 122 aa, Alpaca; identical to SEQ ID NO: 24

SEQ ID NO: 93 - protein, 8 aa, Alpaca
GAVVKPHA

SEQ ID NO: 94 - protein, 8 aa, Alpaca
ISSSGYNT

SEQ ID NO: 95 - protein, 18 aa, Alpaca
ALVLYGGLAGCEAIGTDY

SEQ ID NO: 96 - protein, 125 aa, Alpaca; identical to SEQ ID NO: 28

SEQ ID NO: 97 - protein, 8 aa, Alpaca
GRTFSRYA

SEQ ID NO: 98 - protein, 8 aa, Alpaca
IGGSGVST

SEQ ID NO: 99 - protein, 21 aa, Alpaca
AAGTNFYKGLNYYTGVRNYDS

SEQ ID NO: 100 - protein, 128 aa, Alpaca; identical to SEQ ID NO: 32

SEQ ID NO: 101 - protein, 8 aa, Alpaca
GSTFSFKS

SEQ ID NO: 102 - protein, 7 aa, Alpaca
ISSKGIM

SEQ ID NO: 103 - protein, 14 aa, Alpaca
NVVEVGVGGLVNVY

SEQ ID NO: 104 - protein, 120 aa, Alpaca; identical to SEQ ID NO: 36

SEQ ID NO: 105 - protein, 8 aa, Alpaca
ERTFNKLG

SEQ ID NO: 106 - protein, 8 aa, Alpaca
ISASGLNT

SEQ ID NO: 107 - protein, 15 aa, Alpaca
AAGPRLRVQDPQYDY

SEQ ID NO: 108 - protein, 122 aa, Alpaca; identical to SEQ ID NO: 40

SEQ ID NO: 109 - protein, 8 aa, Alpaca
GRTGNAYR

SEQ ID NO: 110 - protein, 8 aa, Alpaca
ISWNGDST

SEQ ID NO: 111 - protein, 15 aa, Alpaca
AVVKGIGVPPNMYDY

SEQ ID NO: 112 - protein, 122 aa, Alpaca; identical to SEQ ID NO: 44

SEQ ID NO: 113 - protein, 8 aa, Alpaca
GSDFSINA

SEQ ID NO: 114 - protein, 7 aa, Alpaca
ISSGGST

SEQ ID NO: 115 - protein, 10 aa, Alpaca
AILKGATPGY

SEQ ID NO: 116 - protein, 116 aa, Alpaca; identical to SEQ ID NO: 48

SEQ ID NO: 117 - protein, 8 aa, Alpaca
GFTLDVYA

SEQ ID NO: 118 - protein, 8 aa, Alpaca
VSSSGDST

SEQ ID NO: 119 - protein, 16 aa, Alpaca
AAVHPSSCHTKPGFGS

SEQ ID NO: 120 - protein, 123 aa, Alpaca; identical to SEQ ID NO: 52

SEQ ID NO: 121 - protein, 8 aa, Alpaca
GSIFSSDG

SEQ ID NO: 122 - protein, 7 aa, Alpaca
ITSVGNT

SEQ ID NO: 123 - protein, 13 aa, Alpaca
NTKKWRFGSWFDY

SEQ ID NO: 124 - protein, 119 aa, Alpaca; identical to SEQ ID NO: 56

SEQ ID NO: 125 - protein, 8 aa, Alpaca
GSTLDVYA

SEQ ID NO: 126 - protein, 8 aa, Alpaca
ISSSGGST

SEQ ID NO: 127 - protein, 16 aa, Alpaca
AQVHPTSCHTKPGFDS

SEQ ID NO: 128 - protein, 123 aa, Alpaca; identical to SEQ ID NO: 60

SEQ ID NO: 129 - protein, 8 aa, Alpaca
GSTFSINA

SEQ ID NO: 130 - protein, 7 aa, Alpaca
ISSGGST

SEQ ID NO: 131 - protein, 11 aa, Alpaca
NILKGGAAYNY

SEQ ID NO: 132 - protein, 117 aa, Alpaca; identical to SEQ ID NO: 64

SEQ ID NO: 133 - protein, 8 aa, Alpaca
ERTFSKLG

SEQ ID NO: 134 - protein, 8 aa, Alpaca
ISASGLNT

SEQ ID NO: 135 - protein, 15 aa, Alpaca
AAGPRLRVQHPHYDY

SEQ ID NO: 136 - protein, 122 aa, Alpaca; identical to SEQ ID NO: 68

(19) United States
(12) Patent Application Publication
(10) Pub. No.: US 2023/0295343 A1
(43) Pub. Date: Sep. 21, 2023

(54) ANTI-FACTOR XII (FXII) NANOBODY OR ANTIGEN-BINDING FRAGMENT THEREOF, AND USE THEREOF

(71) Applicant: NINGBO COMGEN BIOTECH CO., LTD., Ningbo (CN)

(72) Inventors: Miao WANG, Beijing (CN); Pengfei XU, Beijing (CN)

(73) Assignee: NINGBO COMGEN BIOTECH CO., LTD., Ningbo (CN)

(21) Appl. No.: 18/021,568

(22) PCT Filed: Sep. 21, 2020
(86) PCT No.: PCT/CN2020/116566

§ 371 Date: Feb. 16, 2023

(30) Foreign Application Priority Data
Jun. 16, 2020 (CN) 202010547554.9

(51) Int. Cl. C07K 16/36; C12N 15/63; A61P 9/10

(52) U.S. Cl. CPC C07K 16/36; C12N 15/63; A61P 9/10; C07K 2317/569; C07K 2317/565

(57) ABSTRACT

A nanobody or an antigen-binding fragment thereof against a specific region of a factor XII (FXII) is provided. The nanobody or the antigen-binding fragment thereof binds to FXII through a binding epitope for FXII to block the activation of FXII, and the binding epitope for FXII includes a conformational epitope. A neutralizing antibody for FXII screened by phage display is provided, and it has been found that the neutralizing antibody can treat thrombosis, myocardial ischemia-reperfusion injury (IRI), and vasculitis in mouse arterial thrombosis, rat extracorporeal membrane oxygenation (ECMO), mouse IRI, and vasculitis models.

### CROSS REFERENCE TO THE RELATED APPLICATIONS

This application is the national phase entry of International Application No. PCT/CN2020/116566, filed on Sep. 21, 2020, which is based upon and claims priority to Chinese Patent Application No. 202010547554.9, filed on Jun. 16, 2020, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBYZL022-PKG Sequence Listing.txt, created on 02/14/2023, and is 54,394 bytes in size.

### TECHNICAL FIELD

The present application relates to a nanobody against a factor XII (FXII) heavy chain and a use of FXII as a target for vasculitis.

### BACKGROUND

FXII is a plasma protein with a molecular weight of 80 kDa and is present in the form of an inactive proenzyme at a quiesced state. FXII is a starting molecule of the contact system. FXII present in the form of a proenzyme has the function of limiting the activity of a proteolytic enzyme in plasma. When contacting with some negatively charged molecules such as collagen and polyphosphate, FXII will be activated. Activated FXII (FXIIa) is a protein consisting of two peptide chains linked through disulfide bonds and still has a molecular weight of 80 kDa. Studies have shown that the knockout of an FXII gene or the pharmacological blockade of FXII in mice can significantly inhibit arterial thrombosis, and the deletion or blockade of FXII does not influence bleeding in vivo. At present, FXII is a hot target for the treatment of thrombosis, and some anti-FXII antibodies or FXII inhibitors have entered clinical trials.

At present, the inhibition of thrombosis by antagonizing FXII is mainly divided into two directions: (1) inhibition of FXII activation (an antagonist binds to a functional domain of an FXII heavy chain and occupies a position to inhibit the FXII activation by an FXII activator) and (2) inhibition of FXIIa activity (an antagonist binds to a light chain of activated FXII to inhibit its thrombin activity). It is now clear that thrombosis can be significantly inhibited in both directions. The present application is intended to prepare an FXII-specific antibody for inhibiting FXII activation. There are two reasons for choosing this direction: (1) Inflammation plays a vital role in the occurrence and development of thrombosis and even other cardiovascular diseases (CVDs). A large number of studies have shown that FXII is closely related to the occurrence and development of inflammation, and in recent years, studies have shown that an FXII heavy chain is involved in an FXII-associated inflammatory response. Therefore, an FXII heavy chain-specific antibody can be screened out to inhibit the FXII activation, thereby reducing the production of FXIIa and inhibiting an inflammatory response regulated by FXII. (2) Studies have shown that the inhibitor rHA-infestin-4 for FXIIa can significantly inhibit thrombosis but has an obvious side effect, that is, a plasmin content in the body increases after injection of the inhibitor. Therefore, an FXIIa-specific antibody may not avoid this side effect.

Vasculitis refers to the infiltration of inflammatory cells in vessel walls and around blood vessels and is accompanied by vascular damage, including cellulose deposition, collagen fibrosis, and endothelial cell and muscle cell necrosis. Vasculitis is also known as angiitis. The vasculitis occurrence caused by anaphylaxis-induced immune complex deposition is a common form of vasculitis. The immune complex deposition will destroy small blood vessels in skin tissue, resulting in tissue redness and local bleeding. A traditional treatment method is primarily a combination of a glucocorticoid and an immunosuppressant (such as cyclophosphamide and methotrexate), but this treatment method has a risk of causing immunosuppression or hormonal dysregulation in the body. The present application discovers for the first time that the knockout of an FXII gene or the pharmacological blockade of FXII by a nanobody can significantly improve immune complex-induced vasculitis.

FXII is a protein that includes a multifunctional domain and has a complicated spatial conformation, and a heavy chain of the protein includes six different functional domains, which contribute to the complicated biological functions of FXII. Multiple domains play important roles in the activation of FXII. In a traditional antibody, a complementarity-determining region (CDR) is short, an antigen-antibody binding position is relatively small, and an antigen-binding epitope is a linear epitope. Compared with the traditional antibody, the nanobody has a longer CDR and can bind to a conformational epitope of an antigen. Therefore, the screening of an FXII-specific nanobody has promising prospects.

Currently, the literatures about an antibody for FXII or FXIIa are introduced as follows: an inhibitor rHA-infestin-4 for FXIIa can significantly inhibit arterial thrombosis in mice as reported in Larsson, Magnus, et al. “A factor XIIa inhibitory antibody provides thromboprotection in extracorporeal circulation without increasing bleeding risk.” Science translational medicine 6.222 (2014): 222ra17-222ra17. A genetically engineered antibody for FXIIa can significantly inhibit arterial thrombosis in mice as reported in Hagedorn I, Schmidbauer S, Pleines I, et al. Factor XIIa Inhibitor Recombinant Human Albumin Infestin-4 Abolishes Occlusive Arterial Thrombus Formation Without Affecting Bleeding [J]. Circulation, 2010, 121 (13): 1510-1517. A monoclonal antibody (mAb) for an FXII heavy chain can significantly inhibit arterial thrombosis in baboons as reported in Matafonov, Anton, et al. “Factor XII inhibition reduces thrombus formation in a primate thrombosis model.” Blood, The Journal of the American Society of Hematology 123.11 (2014): 1739-1746. There are currently no published clinical trial data.

### SUMMARY

According to a first aspect of the present application, an anti-FXII nanobody or an antigen-binding fragment thereof is provided to solve the problems in the background. In the present application, a neutralizing antibody for FXII is screened out by phage display, and it has been found that the neutralizing antibody exhibits prominent anti-thrombosis and anti-vasculitis effects in mouse arterial thrombosis and rat extracorporeal membrane oxygenation (ECMO) models.

The anti-FXII nanobody or the antigen-binding fragment thereof binds to FXII through a binding epitope for FXII to block the activation of FXII, and the binding epitope for FXII includes a conformational epitope.

Optionally, the binding epitope of the anti-FXII nanobody or the antigen-binding fragment thereof to FXII is a conformational epitope and can bind to both a fibronectin type II domain and a kringle domain of FXII.

Optionally, the anti-FXII nanobody or the antigen-binding fragment thereof has almost no ability to block an activity of FXIIa.

Optionally, when a molar ratio of the anti-FXII nanobody or the antigen-binding fragment thereof to FXII is 1:(0.1-0.3), a blocking efficiency of the anti-FXII nanobody or the antigen-binding fragment thereof for FXII is no less than 50%.

Optionally, the anti-FXII nanobody or the antigen-binding fragment thereof is derived from an alpaca.

Optionally, the anti-FXII nanobody or the antigen-binding fragment thereof is obtained based on an immune library obtained after immunization of an FXII protein.

Optionally, the anti-FXII nanobody or the antigen-binding fragment thereof is a recombinant antibody.

Optionally, the nanobody is an alpaca nanobody.

Optionally, the recombinant antibody includes a nanobody fused with an immunoglobulin Fc fragment. The fusion with an immunoglobulin Fc fragment significantly increases a half-life of the antibody.

Optionally, based on Kabat Database analysis,

n-1. the anti-FXII nanobody includes a heavy chain CDR-H1 with a sequence shown in SEQ ID NO: 1, a heavy chain CDR-H2 with a sequence shown in SEQ ID NO: 2, and a heavy chain CDR-H3 with a sequence shown in SEQ ID NO: 3; or

n-2. the anti-FXII nanobody includes a heavy chain CDR-H1 with a sequence shown in SEQ ID NO: 5, a heavy chain CDR-H2 with a sequence shown in SEQ ID NO: 6, and a heavy chain CDR-H3 with a sequence shown in SEQ ID NO: 7; or

n-3. the anti-FXII nanobody includes a heavy chain CDR-H1 with a sequence shown in SEQ ID NO: 9, a heavy chain CDR-H2 with a sequence shown in SEQ ID NO: 10, and a heavy chain CDR-H3 with a sequence shown in SEQ ID NO: 11; or

n-4. the anti-FXII nanobody includes a heavy chain CDR-H1 with a sequence shown in SEQ ID NO: 13, a heavy chain CDR-H2 with a sequence shown in SEQ ID NO: 14, and a heavy chain CDR-H3 with a sequence shown in SEQ ID NO: 15; or

n-5. the anti-FXII nanobody includes a heavy chain CDR-H1 with a sequence shown in SEQ ID NO: 17, a heavy chain CDR-H2 with a sequence shown in SEQ ID NO: 18, and a heavy chain CDR-H3 with a sequence shown in SEQ ID NO: 19; or

n-6. the anti-FXII nanobody includes a heavy chain CDR-H1 with a sequence shown in SEQ ID NO: 21, a heavy chain CDR-H2 with a sequence shown in SEQ ID NO: 22, and a heavy chain CDR-H3 with a sequence shown in SEQ ID NO: 23; or

n-7. the anti-FXII nanobody includes a heavy chain CDR-H1 with a sequence shown in SEQ ID NO: 25, a heavy chain CDR-H2 with a sequence shown in SEQ ID NO: 26, and a heavy chain CDR-H3 with a sequence shown in SEQ ID NO: 27; or

n-8. the anti-FXII nanobody includes a heavy chain CDR-H1 with a sequence shown in SEQ ID NO: 29, a heavy chain CDR-H2 with a sequence shown in SEQ ID NO: 30, and a heavy chain CDR-H3 with a sequence shown in SEQ ID NO: 31; or

n-9. the anti-FXII nanobody includes a heavy chain CDR-H1 with a sequence shown in SEQ ID NO: 33, a heavy chain CDR-H2 with a sequence shown in SEQ ID NO: 34, and a heavy chain CDR-H3 with a sequence shown in SEQ ID NO: 35; or

n-10. the anti-FXII nanobody includes a heavy chain CDR-H1 with a sequence shown in SEQ ID NO: 37, a heavy chain CDR-H2 with a sequence shown in SEQ ID NO: 38, and a heavy chain CDR-H3 with a sequence shown in SEQ ID NO: 39; or

n-11. the anti-FXII nanobody includes a heavy chain CDR-H1 with a sequence shown in SEQ ID NO: 41, a heavy chain CDR-H2 with a sequence shown in SEQ ID NO: 42, and a heavy chain CDR-H3 with a sequence shown in SEQ ID NO: 43; or

n-12. the anti-FXII nanobody includes a heavy chain CDR-H1 with a sequence shown in SEQ ID NO: 45, a heavy chain CDR-H2 with a sequence shown in SEQ ID NO: 46, and a heavy chain CDR-H3 with a sequence shown in SEQ ID NO: 47; or

n-13. the anti-FXII nanobody includes a heavy chain CDR-H1 with a sequence shown in SEQ ID NO: 49, a heavy chain CDR-H2 with a sequence shown in SEQ ID NO: 50, and a heavy chain CDR-H3 with a sequence shown in SEQ ID NO: 51; or

n-14. the anti-FXII nanobody includes a heavy chain CDR-H1 with a sequence shown in SEQ ID NO: 53, a heavy chain CDR-H2 with a sequence shown in SEQ ID NO: 54, and a heavy chain CDR-H3 with a sequence shown in SEQ ID NO: 55; or

n-15. the anti-FXII nanobody includes a heavy chain CDR-H1 with a sequence shown in SEQ ID NO: 57, a heavy chain CDR-H2 with a sequence shown in SEQ ID NO: 58, and a heavy chain CDR-H3 with a sequence shown in SEQ ID NO: 59; or

n-16. the anti-FXII nanobody includes a heavy chain CDR-H1 with a sequence shown in SEQ ID NO: 61, a heavy chain CDR-H2 with a sequence shown in SEQ ID NO: 62, and a heavy chain CDR-H3 with a sequence shown in SEQ ID NO: 63; or

n-17. the anti-FXII nanobody includes a heavy chain CDR-H1 with a sequence shown in SEQ ID NO: 65, a heavy chain CDR-H2 with a sequence shown in SEQ ID NO: 66, and a heavy chain CDR-H3 with a sequence shown in SEQ ID NO: 67; and based on IMGT Database analysis,

n-1. the anti-FXII nanobody includes a heavy chain CDR-H1 with a sequence shown in SEQ ID NO: 69, a heavy chain CDR-H2 with a sequence shown in SEQ ID NO: 70, and a heavy chain CDR-H3 with a sequence shown in SEQ ID NO: 71; or

n-2. the anti-FXII nanobody includes a heavy chain CDR-H1 with a sequence shown in SEQ ID NO: 73, a heavy chain CDR-H2 with a sequence shown in SEQ ID NO: 74, and a heavy chain CDR-H3 with a sequence shown in SEQ ID NO: 75; or

n-3. the anti-FXII nanobody includes a heavy chain CDR-H1 with a sequence shown in SEQ ID NO: 77, a heavy chain CDR-H2 with a sequence shown in SEQ ID NO: 78, and a heavy chain CDR-H3 with a sequence shown in SEQ ID NO: 79; or

n-4. the anti-FXII nanobody includes a heavy chain CDR-H1 with a sequence shown in SEQ ID NO: 81, a heavy chain CDR-H2 with a sequence shown in SEQ ID NO: 82, and a heavy chain CDR-H3 with a sequence shown in SEQ ID NO: 83; or

n-5. the anti-FXII nanobody includes a heavy chain CDR-H1 with a sequence shown in SEQ ID NO: 85, a heavy chain CDR-H2 with a sequence shown in SEQ ID NO: 86, and a heavy chain CDR-H3 with a sequence shown in SEQ ID NO: 87; or

n-6. the anti-FXII nanobody includes a heavy chain CDR-H1 with a sequence shown in SEQ ID NO: 89, a heavy chain CDR-H2 with a sequence shown in SEQ ID NO: 90, and a heavy chain CDR-H3 with a sequence shown in SEQ ID NO: 91; or

n-7. the anti-FXII nanobody includes a heavy chain CDR-H1 with a sequence shown in SEQ ID NO: 93, a heavy chain CDR-H2 with a sequence shown in SEQ ID NO: 94, and a heavy chain CDR-H3 with a sequence shown in SEQ ID NO: 95; or

n-8. the anti-FXII nanobody includes a heavy chain CDR-H1 with a sequence shown in SEQ ID NO: 97, a heavy chain CDR-H2 with a sequence shown in SEQ ID NO: 98, and a heavy chain CDR-H3 with a sequence shown in SEQ ID NO: 99; or

n-9. the anti-FXII nanobody includes a heavy chain CDR-H1 with a sequence shown in SEQ ID NO: 101, a heavy chain CDR-H2 with a sequence shown in SEQ ID NO: 102, and a heavy chain CDR-H3 with a sequence shown in SEQ ID NO: 103; or

n-10. the anti-FXII nanobody includes a heavy chain CDR-H1 with a sequence shown in SEQ ID NO: 105, a heavy chain CDR-H2 with a sequence shown in SEQ ID NO: 106, and a heavy chain CDR-H3 with a sequence shown in SEQ ID NO: 107; or

n-11. the anti-FXII nanobody includes a heavy chain CDR-H1 with a sequence shown in SEQ ID NO: 109, a heavy chain CDR-H2 with a sequence shown in SEQ ID NO: 110, and a heavy chain CDR-H3 with a sequence shown in SEQ ID NO: 111; or

n-12. the anti-FXII nanobody includes a heavy chain CDR-H1 with a sequence shown in SEQ ID NO: 113, a heavy chain CDR-H2 with a sequence shown in SEQ ID NO: 114, and a heavy chain CDR-H3 with a sequence shown in SEQ ID NO: 115; or

n-13. the anti-FXII nanobody includes a heavy chain CDR-H1 with a sequence shown in SEQ ID NO: 117, a heavy chain CDR-H2 with a sequence shown in SEQ ID NO: 118, and a heavy chain CDR-H3 with a sequence shown in SEQ ID NO: 119; or

n-14. the anti-FXII nanobody includes a heavy chain CDR-H1 with a sequence shown in SEQ ID NO: 121, a heavy chain CDR-H2 with a sequence shown in SEQ ID NO: 122, and a heavy chain CDR-H3 with a sequence shown in SEQ ID NO: 123; or

n-15. the anti-FXII nanobody includes a heavy chain CDR-H1 with a sequence shown in SEQ ID NO: 125, a heavy chain CDR-H2 with a sequence shown in SEQ ID NO: 126, and a heavy chain CDR-H3 with a sequence shown in SEQ ID NO: 127; or

n-16. the anti-FXII nanobody includes a heavy chain CDR-H1 with a sequence shown in SEQ ID NO: 129, a heavy chain CDR-H2 with a sequence shown in SEQ ID NO: 130, and a heavy chain CDR-H3 with a sequence shown in SEQ ID NO: 131; or

n-17. the anti-FXII nanobody includes a heavy chain CDR-H1 with a sequence shown in SEQ ID NO: 133, a heavy chain CDR-H2 with a sequence shown in SEQ ID NO: 134, and a heavy chain CDR-H3 with a sequence shown in SEQ ID NO: 135.

Optionally, based on Kabat Database analysis,

n-101. the anti-FXII nanobody includes a heavy chain variable region with a sequence shown in SEQ ID NO: 4; or

n-102. the anti-FXII nanobody includes a heavy chain variable region with a sequence shown in SEQ ID NO: 8; or

n-103. the anti-FXII nanobody includes a heavy chain variable region with a sequence shown in SEQ ID NO: 12; or

n-104. the anti-FXII nanobody includes a heavy chain variable region with a sequence shown in SEQ ID NO: 16; or

n-105. the anti-FXII nanobody includes a heavy chain variable region with a sequence shown in SEQ ID NO: 20; or

n-106. the anti-FXII nanobody includes a heavy chain variable region with a sequence shown in SEQ ID NO: 24; or

n-107. the anti-FXII nanobody includes a heavy chain variable region with a sequence shown in SEQ ID NO: 28; or

n-108. the anti-FXII nanobody includes a heavy chain variable region with a sequence shown in SEQ ID NO: 32; or

n-109. the anti-FXII nanobody includes a heavy chain variable region with a sequence shown in SEQ ID NO: 36; or

n-110. the anti-FXII nanobody includes a heavy chain variable region with a sequence shown in SEQ ID NO: 40; or

n-111. the anti-FXII nanobody includes a heavy chain variable region with a sequence shown in SEQ ID NO: 44; or

n-112. the anti-FXII nanobody includes a heavy chain variable region with a sequence shown in SEQ ID NO: 48; or

n-113. the anti-FXII nanobody includes a heavy chain variable region with a sequence shown in SEQ ID NO: 52; or

n-114. the anti-FXII nanobody includes a heavy chain variable region with a sequence shown in SEQ ID NO: 56; or

n-115. the anti-FXII nanobody includes a heavy chain variable region with a sequence shown in SEQ ID NO: 60; or

n-116. the anti-FXII nanobody includes a heavy chain variable region with a sequence shown in SEQ ID NO: 64; or

n-117. the anti-FXII nanobody includes a heavy chain variable region with a sequence shown in SEQ ID NO: 68; and based on IMGT Database analysis,

n-101. the anti-FXII nanobody includes a heavy chain variable region with a sequence shown in SEQ ID NO: 72; or

n-102. the anti-FXII nanobody includes a heavy chain variable region with a sequence shown in SEQ ID NO: 76; or

n-103. the anti-FXII nanobody includes a heavy chain variable region with a sequence shown in SEQ ID NO: 80; or

n-104. the anti-FXII nanobody includes a heavy chain variable region with a sequence shown in SEQ ID NO: 84; or

n-105. the anti-FXII nanobody includes a heavy chain variable region with a sequence shown in SEQ ID NO: 88; or

n-106. the anti-FXII nanobody includes a heavy chain variable region with a sequence shown in SEQ ID NO: 92; or

n-107. the anti-FXII nanobody includes a heavy chain variable region with a sequence shown in SEQ ID NO: 96; or

n-108. the anti-FXII nanobody includes a heavy chain variable region with a sequence shown in SEQ ID NO: 100; or

n-109. the anti-FXII nanobody includes a heavy chain variable region with a sequence shown in SEQ ID NO: 104; or

n-110. the anti-FXII nanobody includes a heavy chain variable region with a sequence shown in SEQ ID NO: 108; or

n-111. the anti-FXII nanobody includes a heavy chain variable region with a sequence shown in SEQ ID NO: 112; or

n-112. the anti-FXII nanobody includes a heavy chain variable region with a sequence shown in SEQ ID NO: 116; or

n-113. the anti-FXII nanobody includes a heavy chain variable region with a sequence shown in SEQ ID NO: 120; or

n-114. the anti-FXII nanobody includes a heavy chain variable region with a sequence shown in SEQ ID NO: 124; or

n-115. the anti-FXII nanobody includes a heavy chain variable region with a sequence shown in SEQ ID NO: 128; or

n-116. the anti-FXII nanobody includes a heavy chain variable region with a sequence shown in SEQ ID NO: 132; or

n-117. the anti-FXII nanobody includes a heavy chain variable region with a sequence shown in SEQ ID NO: 136.

The CDR sequence and heavy chain variable region sequence of the anti-FXII nanobody are shown in Tables 1 and 2 below (The sequences in Table 1 are obtained according to the Kabat Database analysis, and the sequences in Table 2 are obtained according to the IMGT Database analysis).

TABLE 1

| SEQ ID | Clone | | |
|---|---|---|---|
| NO: | No. | Region | Sequence fragment |
| 1 | A4-1 | CDR- | KLGMG |
| | | H1 | |
| 2 | A4-1 | CDR- | VISASGLNTWYGDSVKG |
| | | H2 | |
| 3 | A4-1 | CDR- | GPRLRVQDPQYDY |
| | | H3 | |
| 4 | A4-1 | VH | QVQLVESGGGLVQAGGSLRLSCVASERTFSKLGM |
| | | | GWFRQAPGKEREFVSVISASGLNTWYGDSVKGR |
| | | | FTISRDTATNTIYLQMNSLKPEDTAVYYCAAGPRL |
| | | | RVQDPQYDYWGQGTQVTVSS |
| 5 | A4-24 | CDR- | KLGMG |
| | | H1 | |
| 6 | A4-24 | CDR- | VISASGLNTWTGDSVKG |
| | | H2 | |
| 7 | A4-24 | CDR- | GPRLRVQDPQYDY |
| | | H3 | |
| 8 | A4-24 | VH | QVQLVESGGGLVQAGGSLRLSCVGSERTFSKLG |
| | | | MGWFRQAPGKEREFVSVISASGLNTWTGDSVKG |
| | | | RFTISRDTATNTVYLQMNSLKPEDTAVYYCAAGP |
| | | | RLRVQDPQYDYWGQGTQVTVSS |
| 9 | A4-3 | CDR- | KLGMG |
| | | H1 | |
| 10 | A4-3 | CDR- | VISASGLNTWYGDSVKG |
| | | H2 | |
| 1 | A4-3 | CDR- | GPRLRVQDPQYDY |
| | | H3 | |
| 12 | A4-3 | VH | QVQLVESGGGLVQAGGSLRLSCVGSERTFNKLG |
| | | | MGWFRQAPGKEREFVSVISASGLNTWYGDSVKG |
| | | | RFTISRDTATNTIYLQMNSLKPEDTAVYYCAAGPR |
| | | | LRVQDPQYDYWGQGTQVTVSS |
| 13 | A5-3 | CDR- | KFGAG |
| | | H1 | |
| 14 | A5-3 | CDR- | GLTAIGGTAIYADSVKG |
| | | H2 | |
| 15 | A5-3 | CDR- | GVRQDLRVADYYY |
| | | H3 | |
| 16 | A5-3 | VH | QVQLVESGGGLVQAGGSLRLSCAASGRSIRKFGA |
| | | | GWFRQAPGKEREFVAGLTAIGGTAIYADSVKGRF |
| | | | TISRDNAKNTASLVMNSLRPEDTAVYYCAAGVR |
| | | | QDLRVADYYYWGQGTQVTVS |
| 17 | A5-33 | CDR- | KLGMG |
| | | H1 | |
| 18 | A5-33 | CDR- | VISASGLNTWYGDSVKG |
| | | H2 | |
| 19 | A5-33 | CDR- | GPRLRVQDPQHDY |
| | | H3 | |
| 20 | A5-33 | VH | QVQLVESGGGLVQAGGSLRLSCVASERTFSKLGM |
| | | | GWFRQAPGKEREFVSVISASGLNTWYGDSVKGR |
| | | | FTISRDTATNTVYLQMNSLKPEDTAVYYCAAGPR |
| | | | LRVQDPQHDYWGQGTQDTVSS |
| 21 | A5-44 | CDR- | KLGMG |
| | | H1 | |
| 22 | A5-44 | CDR- | VISATGLNTWYGDSVKG |
| | | H2 | |
| 23 | A5-44 | CDR- | GPRLRVQHPHYDY |
| | | H3 | |
| 24 | A5-44 | VH | QVQLVESGGGLVQAGGSLRLSCVASERTFNKLG |
| | | | MGWFRQAPGKEREFVSVISATGLNTWYGDSVKG |
| | | | RFTISRDTATNTVYLQMNSLKPEDTAVYYCAVGP |
| | | | RLRVQHPHYDYWGQGTQVTVSS |
| 25 | A5-5 | CDR- | PHAIG |
| | | H1 | |
| 26 | A5-5 | CDR- | CISSSGYNTYYAEPVEG |
| | | H2 | |
| 27 | A5-5 | CDR- | VLYGGLAGCEAIGTDY |
| | | H3 | |
| 28 | A5-5 | VH | QVQLVESGGGLVQPGGSLRLSCAESGAVVKPHAI |
| | | | GWFRQVPGKERERVGCISSSGYNTYYAEPVEGRF |
| | | | TISRDNAKNTVYLQMNSLKPEDTAVYYCALVLY |
| | | | GGLAGCEAIGTDYWGKGTLVTVSS |
| 29 | A5-61 | CDR- | RYAMA |
| | | H1 | |
| 30 | A5-61 | CDR- | SIGGSGVSTNYADSVKG |
| | | H2 | |
| 31 | A5-61 | CDR- | GTNFYKGLNYYTGVRNYDS |
| | | H3 | |
| 32 | A5-61 | VH | QVQLVESGGGLVQAGGSLRLSCAASGRTFSRYA |
| | | | MAWFRQAPGKEREFVASIGGSGVSTNYADSVKG |
| | | | RFTISRDNAKNTVTLQMMSLKPEDTGVYYCAAG |
| | | | TNFYKGLNYYTGVRNYDSWGQGTQVSVSS |
| 33 | A5-8 | CDR- | FKSMG |
| | | H1 | |
| 34 | A5-8 | CDR- | AISSKGIMTNSDSVKG |
| | | H2 | |
| 35 | A5-8 | CDR- | VEVGVGGLVNVY |
| | | H3 | |
| 36 | A5-8 | VH | QVQLVESGGGLAQVGGSLRLSCAASGSTFSFKSM |
| | | | GWYREAPGNQRELVAAISSKGIMTNSDSVKGRFT |
| | | | ISRDNAKNTVDLHLNNLKPEDTAVYYCNVVEVG |
| | | | VGGLVNVYWGQGTQVTVSS |
| 37 | N4-13 | CDR- | KLGMG |
| | | H1 | |
| 38 | N4-13 | CDR- | VISASGLNTWYGDSVKG |
| | | H2 | |
| 39 | N4-13 | CDR- | GPRLRVQDPQYDY |
| | | H3 | |
| 40 | N4-13 | VH | QVQLVESGGGLVQAGGSLRLSCVASERTFNKLG |
| | | | MGWFRQAPGKEREFVSVISASGLNTWYGDSVKG |
| | | | RFTISRDTATNTIYLQMNSLKPEDTAVYYCAAGPR |
| | | | LRVQDPQYDYWGQGTQVTVSS |
| 41 | N4-38 | CDR- | AYRMG |
| | | H1 | |
| 42 | N4-38 | CDR- | AISWNGDSTNYADSVKG |
| | | H2 | |
| 43 | N4-38 | CDR- | VKGIGVPPNMYDY |
| | | H3 | |
| 44 | N4-38 | VH | QVQLVESGGGLVQAGGSLRLSCAASGRTGNAYR |
| | | | MGWFRQAPGKEREFVAAISWNGDSTNYADSVKG |
| | | | RFTISRDNAKNTVYLQMNSLKPEDTAVYYCAVV |
| | | | KGIGVPPNMYDYWGQGTQVTVSS |
| 45 | N4-44 | CDR- | INAMG |
| | | H1 | |
| 46 | N4-44 | CDR- | TISSGGSTYYADSVKG |
| | | H2 | |
| 47 | N4-44 | CDR- | LKGATPGY |
| | | H3 | |
| 48 | N4-44 | VH | QVQLVESGGGLVQAGGSLRLSCAASGSDFSINAM |
| | | | GWYRQAPGKERELVATISSGGSTYYADSVKGRFT |
| | | | ISRDNAKNTLYLHMNTLKPEDTAMYYCAILKGAT |
| | | | PGYWGQGTQVTVSS |
| 49 | N4-5 | CDR- | VYAIG |
| | | H1 | |
| in | N4-5 | CDR- | CVSSSGDSTKYADSVKG |
| | | H2 | |
| 5 | N4-5 | CDR- | VHPSSCHTKPGFGS |
| | | H3 | |
| 52 | N4-5 | VH | QVQLVESGGGLVQPGGSLRLSCAASGFTLDVYAI |
| | | | GWFRQAPGKEREGVSCVSSSGDSTKYADSVKGR |
| | | | FTISRDNAKNTVYLQMNILKPEDTAVYYCAAVHP |
| | | | SSCHTKPGFGSWGPGTQVTVSS |
| 53 | N5-1 | CDR- | SDGFG |
| | | H1 | |
| 54 | N5-1 | CDR- | DITSVGNTNYADSVKG |
| | | H2 | |
| 55 | N5-1 | CDR- | KKWRFGSWFDY |
| | | H3 | |
| 56 | N5-1 | VH | QVQLVESGGGLVQAGGSLRLSCAASGSIFSSDGF |
| | | | GWYGQAPGKQRELVADITSVGNTNYADSVKGRF |
| | | | TISRDNAKNTVYLQMNNLKPEDTAVYYCNTKK |
| | | | WRFGSWFDYWGQGTQVTVSS |
| 57 | N5-2 | CDR- | VYAIG |
| | | H1 | |
| 58 | N5-2 | CDR- | CISSSGGSTNYADSVKG |
| | | H2 | |
| 59 | N5-2 | CDR- | VHPTSCHTKPGFDS |
| | | H3 | |
| 60 | N5-2 | VH | QVQLVESGGGLVQPGGSLRLSCAASGSTLDVYAI |
| | | | GWFRQAPGKEREGVSCISSSGGSTNYADSVKGRF |
| | | | TISRDNAKNTVYLQMNILKPEDTAVYHCAQVHPT |
| | | | SCHTKPGFDSWGPGTQVTVAS |
| 61 | N5-20 | CDR- | INAIG |
| | | H1 | |
| 62 | N5-20 | CDR- | AISSGGSTNYADSVKG |
| | | H2 | |
| 63 | N5-20 | CDR- | RFTISRDNAKNTVYLQMNSLKPEDTAVYYCNI |
| | | H3 | |
| 64 | N5-20 | VH | QVQLVESGGGLVQAGGSLRLSCAASGSTFSINAIG |
| | | | WYRQAPGKQRELVAAISSGGSTNYADSVKGRFTI |
| | | | SRDNAKNTVYLQMNSLKPEDTAVYYCNILKGGA |
| | | | AYNYWGQGTQVTVSS |
| 65 | N5-4 | CDR- | KLGMG |
| | | H1 | |
| 66 | N5-4 | CDR- | VISASGLNTWYGDSVKG |
| | | H2 | |
| 67 | N5-4 | CDR- | GPRLRVQHPHYDY |
| | | H3 | |
| 68 | N5-4 | VH | QVQLVESGGGLVQAGGSLRLSCVASERTFSKLGM |
| | | | GWFRQAPGKEREFVSVISASGLNTWYGDSVKGR |
| | | | FTISRDTATNTVYLQMNSLKPEDTAVYYCAAGPR |
| | | | LRVQHPHYDYCGQGTQVTVSS |

TABLE 2

| SEQ ID | Clone | | |
|---|---|---|---|
| NO: | No. | Region | Sequence fragment |
| 69 | A4-1 | CDR- | ERTFSKLG |
| | | H1 | |
| 70 | A4-1 | CDR- | ISASGLNT |
| | | H2 | |
| 71 | A4-1 | CDR- | AAGPRLRVQDPQYDY |
| | | H3 | |
| 72 | A4-1 | VH | QVQLVESGGGLVQAGGSLRLSCVASERTFSKLG |
| | | | MGWFRQAPGKEREFVSVISASGLNTWYGDSVK |
| | | | GRFTISRDTATNTIYLQMNSLKPEDTAVYYCAAG |
| | | | PRLRVQDPQYDYWGQGTQVTVSS |
| 73 | A4-24 | CDR- | ERTFSKLG |
| | | H1 | |
| 74 | A4-24 | CDR- | ISASGLNT |
| | | H2 | |
| 75 | A4-24 | CDR- | AAGPRLRVQDPQYDY |
| | | H3 | |
| 76 | A4-24 | VH | QVQLVESGGGLVQAGGSLRLSCVGSERTFSKLG |
| | | | MGWFRQAPGKEREFVSVISASGLNTWTGDSVK |
| | | | GRFTISRDTATNTVYLQMNSLKPEDTAVYYCAA |
| | | | GPRLRVQDPQYDYWGQGTQVTVSS |
| 77 | A4-3 | CDR- | ERTFNKLG |
| | | H1 | |
| 78 | A4-3 | CDR- | ISASGLNT |
| | | H2 | |
| 79 | A4-3 | CDR- | AAGPRLRVQDPQYDY |
| | | H3 | |
| 80 | A4-3 | VH | QVQLVESGGGLVQAGGSLRLSCVGSERTFNKLG |
| | | | MGWFRQAPGKEREFVSVISASGLNTWYGDSVK |
| | | | GRFTISRDTATNTIYLQMNSLKPEDTAVYYCAAG |
| | | | PRLRVQDPQYDYWGQGTQVTVSS |
| 81 | A5-3 | CDR- | GRSIRKFG |
| | | H1 | |
| 82 | A5-3 | CDR- | LTAIGGTA |
| | | H2 | |
| 83 | A5-3 | CDR- | AAGVRQDLRVADYYY |
| | | H3 | |
| 84 | A5-3 | VH | QVQLVESGGGLVQAGGSLRLSCAASGRSIRKFG |
| | | | AGWFRQAPGKEREFVAGLTAIGGTAIYADSVKG |
| | | | RFTISRDNAKNTASLVMNSLRPEDTAVYYCAAG |
| | | | VRQDLRVADYYYWGQGTQVTVS |
| 85 | A5-33 | CDR- | ERTFSKLG |
| | | H1 | |
| 86 | A5-33 | CDR- | ISASGLNT |
| | | H2 | |
| 87 | A5-33 | CDR- | AAGPRLRVQDPQHDY |
| | | H3 | |
| 88 | A5-33 | VH | QVQLVESGGGLVQAGGSLRLSCVASERTFSKLG |
| | | | MGWFRQAPGKEREFVSVISASGLNTWYGDSVK |
| | | | GRFTISRDTATNTVYLQMNSLKPEDTAVYYCAA |
| | | | GPRLRVQDPQHDYWGQGTQDTVSS |
| 89 | A5-44 | CDR- | ERTFNKLG |
| | | H1 | |
| 90 | A5-44 | CDR- | ISATGLNT |
| | | H2 | |
| 91 | A5-44 | CDR- | AVGPRLRVQHPHYDY |
| | | H3 | |
| 92 | A5-44 | VH | QVQLVESGGGLVQAGGSLRLSCVASERTFNKLG |
| | | | MGWFRQAPGKEREFVSVISATGLNTWYGDSVK |
| | | | GRFTISRDTATNTVYLQMNSLKPEDTAVYYCAV |
| | | | GPRLRVQHPHYDYWGQGTQVTVSS |
| 93 | A5-5 | CDR- | GAVVKPHA |
| | | H1 | |
| 94 | A5-5 | CDR- | ISSSGYNT |
| | | H2 | |
| 95 | A5-5 | CDR- | ALVLYGGLAGCEAIGTDY |
| | | H3 | |
| 96 | A5-5 | VH | QVQLVESGGGLVQPGGSLRLSCAESGAVVKPHA |
| | | | IGWFRQVPGKERERVGCISSSGYNTYYAEPVEGR |
| | | | FTISRDNAKNTVYLQMNSLKPEDTAVYYCALVL |
| | | | YGGLAGCEAIGTDYWGKGTLVTVSS |
| 97 | A5-61 | CDR- | GRTFSRYA |
| | | H1 | |
| 98 | A5-61 | CDR- | IGGSGVST |
| | | H2 | |
| 99 | A5-61 | CDR- | AAGTNFYKGLNYYTGVRNYDS |
| | | H3 | |
| 100 | A5-61 | VH | QVQLVESGGGLVQAGGSLRLSCAASGRTFSRYA |
| | | | MAWFRQAPGKEREFVASIGGSGVSTNYADSVKG |
| | | | RFTISRDNAKNTVTLQMMSLKPEDTGVYYCAA |
| | | | GTNFYKGLNYYTGVRNYDSWGQGTQVSVSS |
| 101 | A5-8 | CDR- | GSTFSFKS |
| | | H1 | |
| 102 | A5-8 | CDR- | ISSKGIM |
| | | H2 | |
| 103 | A5-8 | CDR- | NVVEVGVGGLVNVY |
| | | H3 | |
| 104 | A5-8 | VH | QVQLVESGGGLAQVGGSLRLSCAASGSTFSFKS |
| | | | MGWYREAPGNQRELVAAISSKGIMTNSDSVKGR |
| | | | FTISRDNAKNTVDLHLNNLKPEDTAVYYCNVVE |
| | | | VGVGGLVNVYWGQGTQVTVSS |
| 105 | N4-13 | CDR- | ERTFNKLG |
| | | H1 | |
| 106 | N4-13 | CDR- | ISASGLNT |
| | | H2 | |
| 107 | N4-13 | CDR- | AAGPRLRVQDPQYDY |
| | | H3 | |
| 108 | N4-13 | VH | QVQLVESGGGLVQAGGSLRLSCVASERTFNKLG |
| | | | MGWFRQAPGKEREFVSVISASGLNTWYGDSVK |
| | | | GRFTISRDTATNTIYLQMNSLKPEDTAVYYCAAG |
| | | | PRLRVQDPQYDYWGQGTQVTVSS |
| 109 | N4-38 | CDR- | GRTGNAYR |
| | | H1 | |
| 110 | N4-38 | CDR- | ISWNGDST |
| | | H2 | |
| 111 | N4-38 | CDR- | AVVKGIGVPPNMYDY |
| | | H3 | |
| 112 | N4-38 | VH | QVQLVESGGGLVQAGGSLRLSCAASGRTGNAYR |
| | | | MGWFRQAPGKEREFVAAISWNGDSTNYADSVK |
| | | | GRFTISRDNAKNTVYLQMNSLKPEDTAVYYCAV |
| | | | VKGIGVPPNMYDYWGQGTQVTVSS |
| 113 | N4-44 | CDR- | GSDFSINA |
| | | H1 | |
| 114 | N4-44 | CDR- | ISSGGST |
| | | H2 | |
| 115 | N4-44 | CDR- | AILKGATPGY |
| | | H3 | |
| 116 | N4-44 | VH | QVQLVESGGGLVQAGGSLRLSCAASGSDFSINA |
| | | | MGWYRQAPGKERELVATISSGGSTYYADSVKGR |
| | | | FTISRDNAKNTLYLHMNTLKPEDTAMYYCAILK |
| | | | GATPGYWGQGTQVTVSS |
| 117 | N4-5 | CDR- | GFTLDVYA |
| | | H1 | |
| 118 | N4-5 | CDR- | VSSSGDST |
| | | H2 | |
| 119 | N4-5 | CDR- | AAVHPSSCHTKPGFGS |
| | | H3 | |
| 120 | N4-5 | VH | QVQLVESGGGLVQPGGSLRLSCAASGFTLDVYAI |
| | | | GWFRQAPGKEREGVSCVSSSGDSTKYADSVKG |
| | | | RFTISRDNAKNTVYLQMNILKPEDTAVYYCAAV |
| | | | HPSSCHTKPGFGSWGPGTQVTVSS |
| 121 | N5-1 | CDR- | GSIFSSDG |
| | | H1 | |
| 122 | N5-1 | CDR- | ITSVGNT |
| | | H2 | |
| 123 | N5-1 | CDR- | NTKKWRFGSWFDY |
| | | H3 | |
| 124 | N5-1 | VH | QVQLVESGGGLVQAGGSLRLSCAASGSIFSSDGF |
| | | | GWYGQAPGKQRELVADITSVGNTNYADSVKGR |
| | | | FTISRDNAKNTVYLQMNNLKPEDTAVYYCNTKK |
| | | | WRFGSWFDYWGQGTQVTVSS |
| 125 | N5-2 | CDR- | GSTLDVYA |
| | | H1 | |
| 126 | N5-2 | CDR- | ISSSGGST |
| | | H2 | |
| 127 | N5-2 | CDR- | AQVHPTSCHTKPGFDS |
| | | H3 | |
| 128 | N5-2 | VH | QVQLVESGGGLVQPGGSLRLSCAASGSTLDVYAI |
| | | | GWFRQAPGKEREGVSCISSSGGSTNYADSVKGR |
| | | | FTISRDNAKNTVYLQMNILKPEDTAVYHCAQVH |
| | | | PTSCHTKPGFDSWGPGTQVTVAS |
| 129 | N5-20 | CDR- | GSTFSINA |
| | | H1 | |
| 130 | N5-20 | CDR- | ISSGGST |
| | | H2 | |
| 131 | N5-20 | CDR- | NILKGGAAYNY |
| | | H3 | |
| 132 | N5-20 | VH | QVQLVESGGGLVQAGGSLRLSCAASGSTFSINAI |
| | | | GWYRQAPGKQRELVAAISSGGSTNYADSVKGRF |
| | | | TISRDNAKNTVYLQMNSLKPEDTAVYYCNILKG |
| | | | GAAYNYWGQGTQVTVSS |
| 133 | N5-4 | CDR- | ERTFSKLG |
| | | H1 | |
| 134 | N5-4 | CDR- | ISASGLNT |
| | | H2 | |
| 135 | N5-4 | CDR- | AAGPRLRVQHPHYDY |
| | | H3 | |
| 136 | N5-4 | VH | QVQLVESGGGLVQAGGSLRLSCVASERTFSKLG |
| | | | MGWFRQAPGKEREFVSVISASGLNTWYGDSVK |
| | | | GRFTISRDTATNTVYLQMNSLKPEDTAVYYCAA |
| | | | GPRLRVQHPHYDYCGQGTQVTVSS |

According to a second aspect of the present application, a nucleic acid encoding the anti-FXII nanobody or the antigen-binding fragment thereof described above is provided.

According to a third aspect of the present application, a vector including the nucleic acid that is effectively linked to an appropriate promoter sequence is provided.

According to a fourth aspect of the present application, a prokaryotic cell, cell line, yeast cell, or viral system including the vector is provided.

According to a fifth aspect of the present application, a method for preparing the antibody or the antigen-binding fragment thereof described above is provided, including:

cultivating the prokaryotic cell, cell line, yeast cell, or viral system under conditions suitable for expression of the antibody, and isolating and purifying the antibody from a culture supernatant.

According to a sixth aspect of the present application, a use of the antibody or the antigen-binding fragment thereof in medicine is provided.

According to a seventh aspect of the present application, a use of the antibody or the antigen-binding fragment thereof in the preparation of an antithrombotic drug is provided.

According to an eighth aspect of the present application, an antithrombotic use of the antibody or the antigen-binding fragment thereof in an artificial medical device in contact with blood is provided.

According to a ninth aspect of the present application, an antithrombotic use of the antibody or the antigen-binding fragment thereof in ECMO is provided.

According to a tenth aspect of the present application, a use of the antibody or the antigen-binding fragment thereof in the preparation of an antivasculitis drug is provided.

According to an eleventh aspect of the present application, a use of the antibody or the antigen-binding fragment thereof in the preparation of an anti-cardiac ischemia-reperfusion injury (IRI) drug is provided.

According to a twelfth aspect of the present application, a pharmaceutical composition including the antibody or the antigen-binding fragment thereof is provided.

According to a thirteenth aspect of the present application, a target for inhibiting vasculitis is provided, and the target is FXII.

According to a fourteenth aspect of the present application, a drug for inhibiting vasculitis is provided, and the drug is an antibody for inhibiting FXII.

According to a fifteenth aspect of the present application, a pharmaceutical composition for inhibiting vasculitis is provided, and the pharmaceutical composition is an antibody for inhibiting FXII.

In the present application, the “recombinant antibody” refers to an antibody obtained through artificial modification or recombinant expression based on a natural mAb, a nanobody, or another single epitope-recognizing antibody.

In the present application, the “conformational epitope” refers to the fibronectin type II domain and kringle domain of FXII.

Possible beneficial effects of the present application:

1) The present application provides an anti-FXII nanobody or an antigen-binding fragment thereof. Compared with the traditional antibodies, the nanobody has a longer CDR, and a binding epitope of the nanobody to FXII is a conformational epitope and can bind to both of the two epitopes with important functions of FXII.

2) The present application provides an anti-FXII nanobody or an antigen-binding fragment thereof, which can block FXII activity. The treatment with the anti-FXII nanobody or the antigen-binding fragment thereof can significantly prolong the time of FeCl₃-induced carotid arterial thrombosis in mice and the time of laser-induced cremaster arteriolar thrombosis in mice, and can significantly reduce the thrombus deposition on the oxygenator membrane during ECMO in rats.

3) The present application provides an anti-FXII nanobody or an antigen-binding fragment thereof, where the anti-FXII nanobody is an immunoglobulin-modified nanobody, Fc is linked in series to a C-terminus of the nanobody, and a half-life of the anti-FXII nanobody is extended to 6 h.

4) The present application provides an anti-FXII nanobody or an antigen-binding fragment thereof, and the knockout of an FXII gene or the treatment with the nanobody, of which C-terminus is tandem to Fc, can significantly improve the immune complex-induced cutaneous vasculitis in mice.

### BRIEF DESCRIPTION OF THE DRAWINGS

FIGS. 1A-1B show the preparation and identification results of a nanobody, where FIG. 1A shows the sodium dodecyl sulfate-polyacrylamide gel electrophoresis (SDS-PAGE) results of the nanobody (Nb) and Fc linked in series (Nb-Fc); and FIG. 1B shows the western blot (WB) identification results of the nanobody (Nb) and Fc linked in series (Nb-Fc).

FIGS. 2A-2D show the titer detection results of screened nanobodies, where FIG. 2A, FIG. 2B, FIG. 2C, and FIG. 2D are for nanobodies corresponding to different clone numbers.

FIGS. 3A-3C show the efficiency of nanobodies to block the activation of FXII, where FIG. 3A, FIG. 3B, and FIG. 3C are for nanobodies corresponding to different clone numbers.

FIG. 4 shows the efficiency of nanobodies to block FXIIa.

FIG. 5 shows the determination results of affinity of the nanobody N4-38 to FXII.

FIG. 6 shows the binding of the nanobody N4-38 (N38) to natural FXII and denatured FXII.

FIG. 7 shows the binding of the nanobody N4-38 to different functional domains of FXII.

FIGS. 8A-8B show the half-life and activity changes of the nanobody N38 (N4-38) after Fc is linked in series, where FIG. 8A shows the half-life results of N38 and N38-Fc; and FIG. 8B shows the efficiency of N38-Fc to block the activation of FXII.

FIGS. 9A-9B show that the treatment with the nanobody N38-Fc can significantly prolong the time of carotid arterial thrombosis in mice and the time of cremasteric arterial thrombosis in mice, where FIG. 9A shows an effect of N38-Fc treatment on FcCl₃-induced carotid arterial thrombosis; and FIG. 9B shows the effect of N38-Fc treatment on laser-induced cremaster arteriolar thrombosis.

FIGS. 10A-10F show the inhibition of the nanobody N38-Fc on the thrombus deposition on the oxygenator membrane and the increase of peripheral blood cells and inflammatory factors during ECMO in rats, where FIG. 10A shows an effect of N38-Fc treatment on the thrombus deposition on the oxygenator membrane during ECMO in rats; FIG. 10B shows the composition of a thrombus on the oxygenator membrane of ECMO; FIG. 10C shows the protein content on the oxygenator membrane of ECMO in different groups; FIG. 10D shows an effect of N38-Fc treatment on the change of peripheral blood leucocytes before and after ECMO in rats; FIG. 10E shows an effect of N38-Fc treatment on the change of peripheral erythrocytes before and after ECMO in rats; and FIG. 10F shows an effect of N38-Fc treatment on the change of TNF-αlevel in peripheral blood before and after ECMO in rats.

FIGS. 11A-11C show that the knockout of an FXII gene and the N38-Fc treatment can significantly improve the immune complex-induced vasculitis, where FIG. 11A shows the representative images of immune complex-induced inflammatory spots; FIG. 11B shows the diameters of immune complex-induced inflammatory spots; and FIG. 11C shows the hemoglobin contents in immune complex-induced inflammatory spots.

FIGS. 12A-12F show the influence of N38-Fc intervention on infarct size (IS) inarea at risk (AAR), where FIG. 12A shows the representative Evans Blue-TTC staining results in a control group and an N38-Fc treatment group; FIG. 12B shows a ratio of IS to ischemic area in each of the control group and the N38-Fc treatment group; FIG. 12C shows a ratio of ischemic area to a sum of ischemic area and non-ischemic area in each of the control group and the N38-Fc treatment group; FIG. 12D shows the representative echocardiograph of cardiac functions in the control group and the N38-Fc treatment group; FIG. 12E shows the changes of ejection fraction (EF) in the control group and the N38-Fc treatment group; and FIG. 12F shows the changes of fractional shortening (FS) in the control group and the N38-Fc treatment group.

### DETAILED DESCRIPTION OF THE EMBODIMENTS

The present application will be described in detail below with reference to examples, but the present application is not limited to these examples.

Unless otherwise specified, the raw materials in the examples of the present application are all purchased from commercial sources.

Analysis methods in the examples of the present application are as follows: DNA amplification is conducted with an ABI Veriti PCR instrument.

An OD value is determined with a Tecan Infinite M200 Pro microplate reader.

### Example 1 Preparation of an FXII Nanobody

Human FXII (Haematologic Technologies Inc, 800 μg/animal) was mixed with a complete Freund's adjuvant (CFA) (a concentration of FXII in the CFA was 0.50 mg/mL), and a resulting mixture was emulsified and then used to immunize an alpaca for the first time; and 21 d, 35 d, and 49 d later, the alpaca was immunized with a mixture of human FXII (400 μg/animal) and incomplete freund's adjuvant (IFA) (sigma) (a concentration of FXII in the IFA was 0.50 mg/mL) for the second time, the third time, and the fourth time, respectively. 7 d after the last immunization, peripheral blood was collected from the alpaca, and a titer of an anti-FXII antibody in the peripheral blood was detected.

When the titer met criteria, lymphocytes in the peripheral blood were isolated, RNA was extracted and reverse-transcribed to obtain a cDNA fragment, and then a nanobody fragment was obtained through nested PCR. The nanobody fragment was inserted into a phage display vector pHEN1 to construct an anti-FXII phage display library, which was directly used for affinity screening of specific phages. The library was screened using human FXII protein for five rounds through liquid phase screening, then the clones were randomly picked from plates for screening and eluting phages, and positive clones were identified by PHAGE-ELISA and sent for sequencing to obtain different nanobody sequences. A nanobody sequence was linked to a prokaryotic expression vector PET26b, a constructed plasmid was transformed into Escherichia coli (E. coli), and isopropylthiogalactoside (IPTG) was added at 0.25 mmol/L to induce the expression of a nanobody. Bacteria were collected and subjected to ultrasonic disruption, a resulting mixture was centrifuged, and a resulting supernatant was subjected to AKTA purifier Ni affinity chromatography for purification to obtain a nanobody protein with a purity of higher than 85%.

### Example 2

The titer detection method in Example 1 was as follows: An ELISA plate was coated with human FXII at a protein concentration of 1 μg/mL overnight at 4° C.; then the ELISA plate was washed with PBST buffer and blocked with 5% skimmed milk powder at room temperature for 2 h; the ELISA plate was washed with PBST buffer (which was prepared with 1 L of PBS and 500 μL of Tween 20), then nanobodies at different dilutions were added, and the ELISA plate was incubated for 2 h at room temperature; the ELISA plate was washed with PBST buffer, then an anti-horseradish peroxidase (HRP)-labeled 6×His tag antibody (Abcam) was added, and the ELISA plate was incubated for 1 h at room temperature in the dark; the ELISA plate was washed with PBST buffer, a tetramethylbenzidine (TMB) (R&D Systems) substrate solution was added, and a chromogenic reaction was conducted for 5 min to 10 min; and a stop solution (sulfuric acid with a concentration of 2 M) was added, and an OD value of each well was determined at 450 nm by a microplate reader.

Titer detection results are shown in FIGS. 2A-2D. In FIGS. 2A-2D, “A5-8” or the like corresponds to a clone number in Table 1, and “BSA” refers to a negative control that cannot bind to a nanobody. The results in FIGS. 2A-2D show that a titer of each of N4-38, N4-44, and A5-8 can reach 220. The titer here is defined as a lowest dilution of an antibody at which the antibody can bind to an antigen and an OD value of an antibody group is 2 times higher than an OD value of a negative control group.

### Example 3

1 μg of human FXII was thoroughly mixed with 140 μl of the nanobody at different concentrations, and a resulting mixture was incubated at 37° C. for 30 min; then 20 μl of ellagic acid (final concentration: 4 μg/mL) was added, and a resulting mixture was thoroughly mixed and incubated at 37° C. for 10 min; then 40 μl of S-2302 (kallikrein chromogenic substrate, 4 mmol/mL) was added, and a resulting mixture was thoroughly mixed and incubated at 37° C. for 15 min; and finally 40 μl of 20% acetic acid was added, and an OD value was detected at 405 nm.

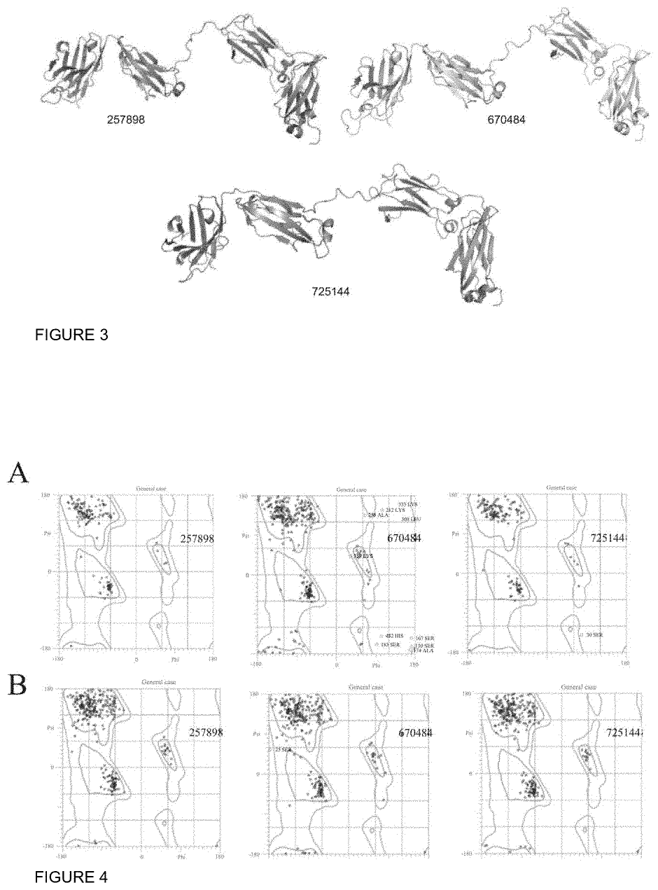

FIGS. 3A-3C show the efficiency of the nanobody to block the activation of FXII. In FIGS. 3A-3C, “N4-38” or the like corresponds to a clone number in Table 1. The results show that N4-38 exhibits excellent blocking activity, and a dilution concentration corresponding to this blocking activity is determined to be a maximum dilution concentration.

### Example 4

1 μg of human FXIIa was thoroughly mixed with 140 μl of the nanobody at different concentrations, and a resulting mixture was incubated at 37° C. for 30 min; then 40 μl of S-2302 (4 mmol/mL) was added, and a resulting mixture was thoroughly mixed and incubated at 37° C. for 15 min; and finally 40 μl of 20% acetic acid was added, and an OD value was detected at 405 nm.

FIG. 4 shows the efficiency of the nanobody to block FXIIa. In FIG. 4, “N4-38” or the like corresponds to a clone number in Table 1, and “PBS” refers to a buffer for dissolving the nanobody. The results show that the screened nanobody cannot block the activity of FXIIa.

### Example 5

(1) A Fortebio Octet special solution M (a PBST solution including BSA with a mass fraction of 0.5%) was prepared, filtered through a 0.22 μm filter membrane, and stored at 4° C. for later use; (2) a biotinylated FXII protein was diluted to 10 μg/mL, a nanobody N4-38 was diluted to 50 μg/mL, 20 μg/mL, and 10 μg/mL, and resulting samples each were added; (3) a biosensor was placed in a corresponding clean well of a spare box, and 200 mL of the Fortebio Octet special solution M was added to each well corresponding to the biosensor; (4) a program was set as follows: first stage Baseline: 60 s, second stage Loading: 200 s, third stage Baseline 2: 100 s, fourth stage Association: 300 s, fourth stage Dissociation: 300 s, and reaction temperature during the whole process: 37° C.; and (5) after a reaction was completed, data analysis was conducted by software.

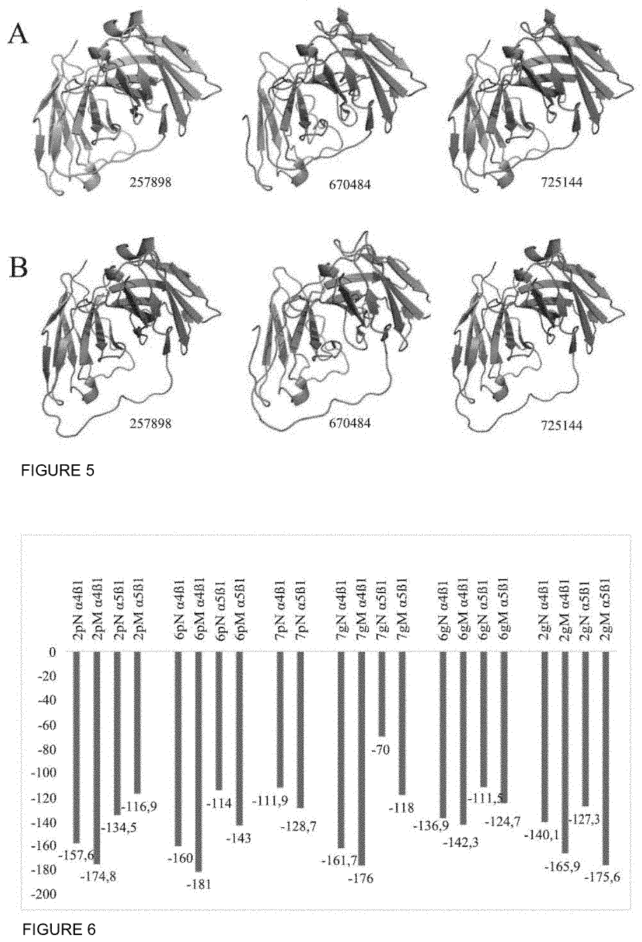

The three curves from top to bottom in FIG. 5 correspond to 50 μg/mL, 20 μg/mL, and 10 μg/mL, respectively. FIG. 5 shows the determination results of affinity of the nanobody N4-38 to FXII, and the results show that an affinity KD of N4-38 to FXII is 3.07×10⁻⁹ (M).

### Example 6

An NC membrane was taken, and 5 mg of natural human FXII, denatured human FXII (which was treated with 100 Mm DTT for 10 min and then boiled at a high temperature for 10 min), and BSA were added dropwise on the NC membrane; the NC membrane was dried and then blocked with 5% skimmed milk powder at room temperature for 2 h; then the N4-38 antibody (1:10,000) was added, and the NC membrane was incubated at room temperature for 2 h; the NC membrane was washed with PBST, an HRP-labeled anti-6HIS tag antibody (1:1,000) was added, and the NC membrane was incubated at room temperature for 1 h; and the NC membrane was washed with PBST, and an ECL luminescent solution was added for exposure.

FIG. 6 shows the binding of the nanobody N4-38 (N38) to natural FXII and denatured FXII. The results show that N4-38 can bind to both natural and denatured FXII, but a binding amount of N4-38 to natural FXII is significantly higher than a binding amount of N4-38 to denatured FXII, indicating that a binding epitope of FXII to N4-38 is a conformational epitope.

### Example 7

A 15% sodium dodecyl sulfate-polyacrylamide gel electrophoresis (SDS-PAGE) gel was prepared, then the treated fibronectin domain type II (FBII), epidermal-growth-factor-like domain (EGFI), fibronectin domain type I (FBI), second epidermal-growth-factor-like domain (EFGII), kringle (KNG), and proline rich region (PRO) protein samples were spotted into the gel, and electrophoresis was conducted. When the samples reached a bottom of the gel, the SDS-PAGE gel was removed and placed in a membrane transfer instrument for membrane transfer. After the membrane transfer was completed, an NC membrane was taken out and blocked with 5% skimmed milk powder at room temperature for 2 h; then the N4-38 antibody (a volume ratio of the antibody to an antibody dilution was 1:10,000) was added to the NC membrane, and the NC membrane was incubated at room temperature for 2 h; the NC membrane was washed with PBST, an HRP-labeled anti-6×HIS tag antibody (a volume ratio of the antibody to an antibody dilution was 1:1,000) was added, and the NC membrane was incubated at room temperature for 1 h; and the NC membrane was washed with PBST, and an ECL solution was added for exposure.

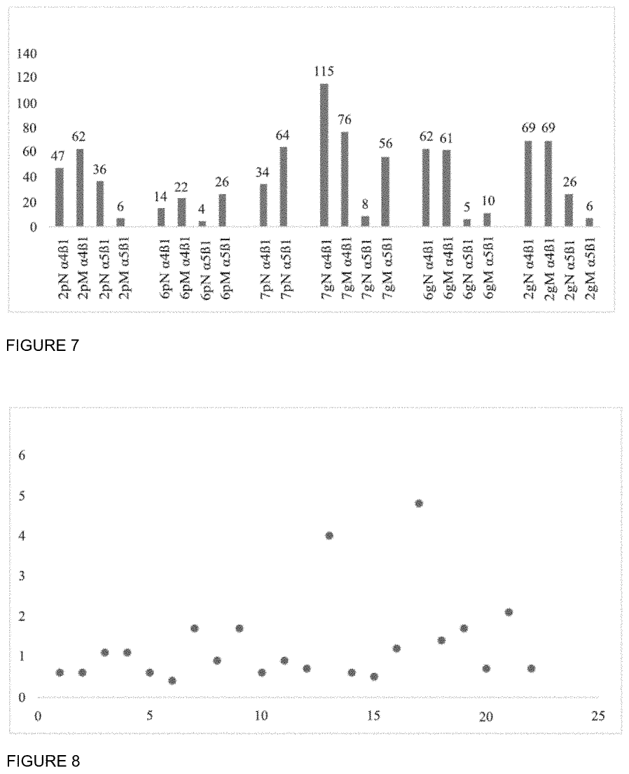

FIG. 7 shows the binding of the nanobody N4-38 to different functional domains of FXII, and the results show that the nanobody specifically binds to the FBII and KNG domains of FXII.

### Example 8

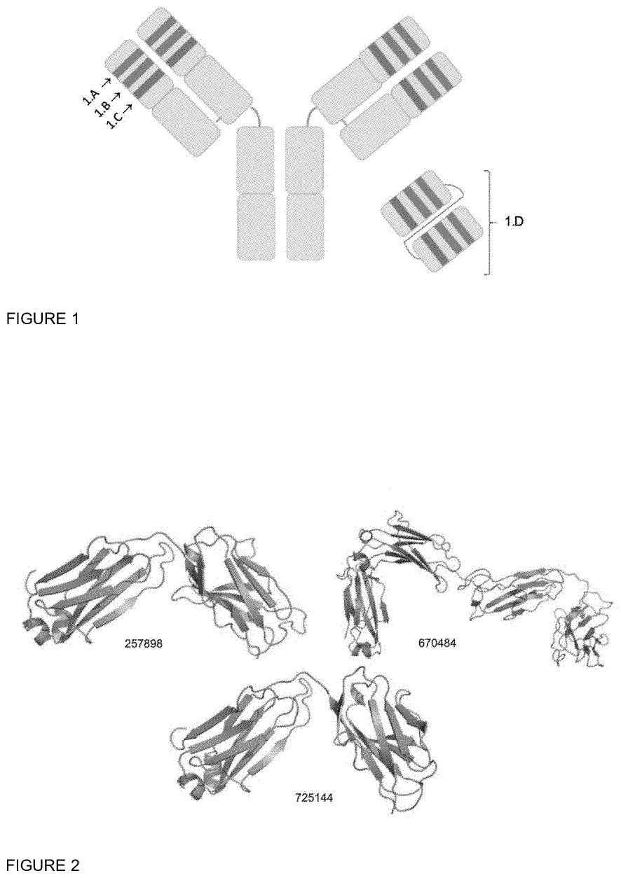

A gene fragment for an antibody fragment obtained by linking a Fc fragment of human IgG through 7 repeated GS linkers in series to a C terminus of the nanobody N38 was prepared through gene synthesis, and the gene fragment was inserted into a prokaryotic expression vector PET26b; and a constructed plasmid was transformed into E. coli, and IPTG was added to induce the expression. Bacteria were collected and subjected to ultrasonic disruption, a resulting mixture was centrifuged, and a resulting supernatant was subjected to AKTA purifier Ni affinity chromatography for purification to obtain a N38-Fc protein. FIGS. 1A-1B show the preparation and identification results of the nanobody, where A shows the SDS-PAGE results of the nanobody (Nb) and Fc linked in series (Nb-Fc); and B shows the WB identification results of the nanobody (Nb) and Fc linked in series (Nb-Fc). FIGS. 1A-1B show the successful preparation of the anti-FXII nanobody with high purity.

(A) The N38 and N38-Fc proteins each were injected intravenously into C57BL/6 mice (1 mg/kg), and 30 min, 1 h, 2 h, 3 h, 4 h, 5 h, 6 h, 7 h, 8 h, and 16 h after the injection, peripheral blood was collected from the mice, and plasma was isolated through centrifugation; 50 μl of the plasma was taken and mixed with 50 μl of a coating solution, and a resulting mixture was added to an ELISA plate and incubated overnight at 4° C.; the ELISA plate was washed with PBST and then blocked with 5% skimmed milk powder at room temperature for 2 h; the ELISA plate was washed with PBST, then an anti-HRP-labeled 6×His tag antibody (Abeam) was added, and the ELISA plate was incubated for 1 h at room temperature in the dark; the ELISA plate was washed with PBST, a TMB substrate solution was added, and a chromogenic reaction was conducted for 5 min to 10 min; and a stop solution was added, and an OD value of each well was determined at 450 nm by a microplate reader.

(B) 1 μg of human FXII was thoroughly mixed with 140 μl of N38 or N38-Fc at different concentrations, and a resulting mixture was incubated at 37° C. for 30 min; then 20 μl of ellagic acid (final concentration: 4 μg/mL) was added, and a resulting mixture was thoroughly mixed and incubated at 37° C. for 10 min; then 40 μl of S-2302 (4 mmol/mL) was added, and a resulting mixture was thoroughly mixed and incubated at 37° C. for 15 min; and finally 40 μl of 20% acetic acid was added, and an OD value was detected at 405 nm.

FIGS. 8A-8B show the half-life and activity changes of the nanobody N38 (N4-38) after Fc is linked in series, where FIG. 8A shows the half-life results of N38 and N38-Fc; FIG. 8B shows the efficiency of N38-Fc to block the activation of FXII; and the “control” refers to the injection of PBS of a same volume for dissolving N38 (N4-38) with Fc linked in series. FIGS. 8A-8B show that the linkage of Fc in series to the nanobody can significantly prolong the half-life of the nanobody without significantly affecting the biological activity of the nanobody.

### Example 9

(A) 8-week-old male C57BL/6 mice were selected, grouped, and intraperitoneally injected with the N38-Fc protein at different doses. 30 min later, the mice were anesthetized with pentobarbital (80 mg/kg), and the left carotid artery was collected; a round filter paper (r=1.0 mm) was placed above the blood vessel, then 0.5 μL of 7.5% FeCl₃ (sigma) was added dropwise on the filter paper, and the filter paper was removed 3 min later; and a Doppler sonography blood flow monitor (Transonic Systems Inc.) was used to measure a blood flow of the carotid artery. (B) 8-week-old male C57BL/6 mice were selected, grouped, and intraperitoneally injected with the N38-Fc protein at different doses. 30 min later, the mice are anesthetized with pentobarbital (80 mg/kg) and simultaneously injected with 5% dextran-FITC (500 mg/kg); a cremaster muscle was collected, and a blood vessel was subjected to thermal damage with 488 nm laser (power: 5 mw) of a confocal microscope; and a time of cremasteric arterial thrombosis was observed.

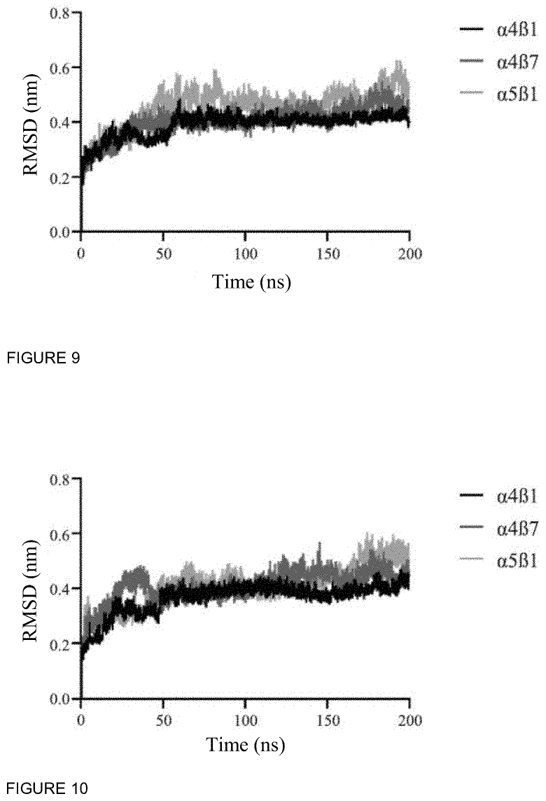

FIG. 9A shows an effect of N38-Fc treatment on FcCl₃-induced carotid arterial thrombosis, and FIG. 9B shows an effect of N38-Fc treatment on laser-induced cremasteric arterial thrombosis, where the “control” refers to the injection of PBS of a same volume for dissolving N38-Fc. FIGS. 9A-9B show that the nanobody N38-Fc treatment significantly prolongs a time of carotid arterial thrombosis and a time of cremasteric arterial thrombosis in mice, indicating that the nanobody N38-Fc treatment can significantly inhibit the arterial thrombosis.

### Example 10

An ECMO system composed of a peristaltic pump connected to a silicone tube, a 10 mL syringe, and a customized small-volume oxygenator (500 cm² gas exchange membrane, Dongguan Kewei Medical Equipment Co., Ltd.) was adopted. The entire circuit was pre-filled with 8 mL of a 6% hydroxyethyl starch (HES) injection. The entire circuit was free of heparin coating. A 400 g male SD rat was anesthetized with an isoflurane gas, subjected to endotracheal intubation, connected to a ventilator, and then injected with heparin (500 U/kg) alone or heparin (500 U/kg) in combination with N38-Fc (2 mg/kg). The femoral artery and jugular vein of the rat were collected, and the ECMO system was connected to each of the femoral artery and jugular vein of the rat through a catheter to form a complete rat ECMO circuit. An ACT value was detected once and circulation was conducted for 2 h every 30 min. 200 μL of peripheral blood was collected from the rat at 5 min and 2 h of the circulation, 30 μL of whole blood was collected, and a small animal whole blood cell counter (HEMAVET 950FS) was used to measure a change of blood cells during an ECMO process; and the remaining peripheral blood was centrifuged to obtain plasma, and a change of TNF-αlevel in rat peripheral blood before and after the circulation was detected by a rat TNF-αELISA detection kit (RayBiotech). After the 2 h of circulation was completed, the oxygenator membrane was taken out, rinsed with PBS, and observed by scanning electron microscopy (SEM) (as shown in FIGS. 10A-10B). In order to analyze the thrombi deposited on the oxygenator membrane, the oxygenator membrane was placed in 1 M NaOH for 24 h, then taken out, and observed under a microscope, and it showed that the cell protein on the oxygenator membrane was completely dissolved; and the protein contents in a NaOH eluate were determined by BCA method.

FIGS. 10A-10C show that the nanobody N38-Fc inhibits the thrombus deposition on the oxygenator membrane during the ECMO process in the rat, indicating that the nanobody N38-Fc intervention significantly inhibits the thrombus deposition on the oxygenator membrane of ECMO. In addition, FIGS. 10D-10F show that the nanobody N38-Fc intervention significantly reduces the increase levels of peripheral blood TNF-α, leucocytes, and erythrocytes during the ECMO process, indicating that the nanobody N38-Fc intervention significantly inhibits the inflammatory response and fluid loss during the ECMO process. The “Control” in the figure refers to the injection of PBS of a same volume for dissolving N38-Fc.

### Example 11

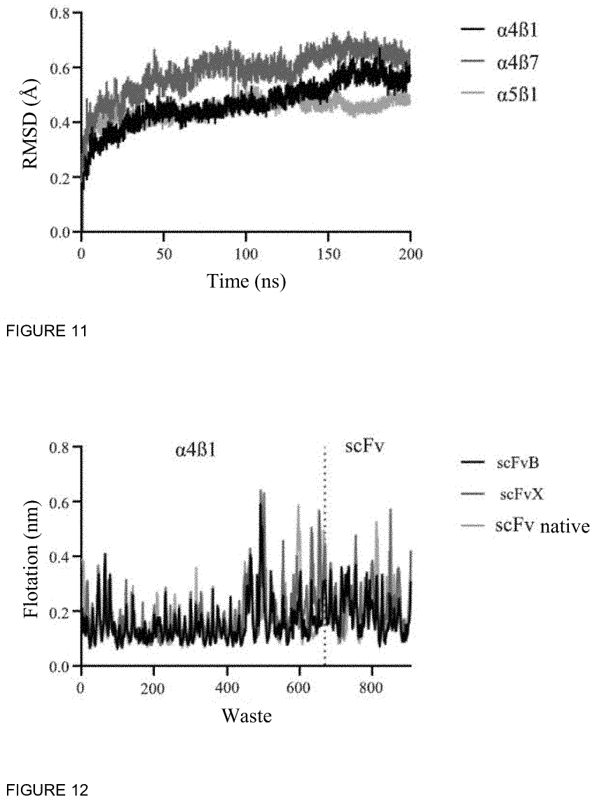

8-week-old C57BL/6 mice or FXII-knockout mice were selected; and the C57BL/6 mice each were treated with N38-Fc (2 mg/kg) and an isotype control thereof and then anesthetized with pentobarbital, hair on the back was removed with depilatory paste, and then the mice each were intravenously injected with BSA (7511 g/g, sigma) and then immediately injected with 20 μL of an anti-BSA polyclonal antibody (pAb) (60 sigma) intracutaneously through the dorsal skin. 4 h later, the mice were euthanized, the dorsal skin was collected, and a diameter of an inflammatory spot at an inner side of the skin was measured (results were shown in FIGS. 11A-11B); a mass of the skin was measured; an RIPA lysis buffer was added to the skin tissue, and the skin tissue was ground; a resulting mixture was centrifuged, and a resulting supernatant was collected; and a hemoglobin content was detected with a Hemoglobin Kit (Abcam) (results were shown in FIG. 11C).

FIGS. 11A-11C show that the knockout of an FXII gene and the N38-Fc treatment can significantly improve the immune complex-induced vasculitis, indicating that FXII is involved in an immune complex-induced vasculitic injury, and the targeted inhibition of FXII can now improve the immune complex-induced vasculitic injury. The “Control” in the figure refers to the injection of PBS of a same volume for dissolving N38-Fc.

### Example 12

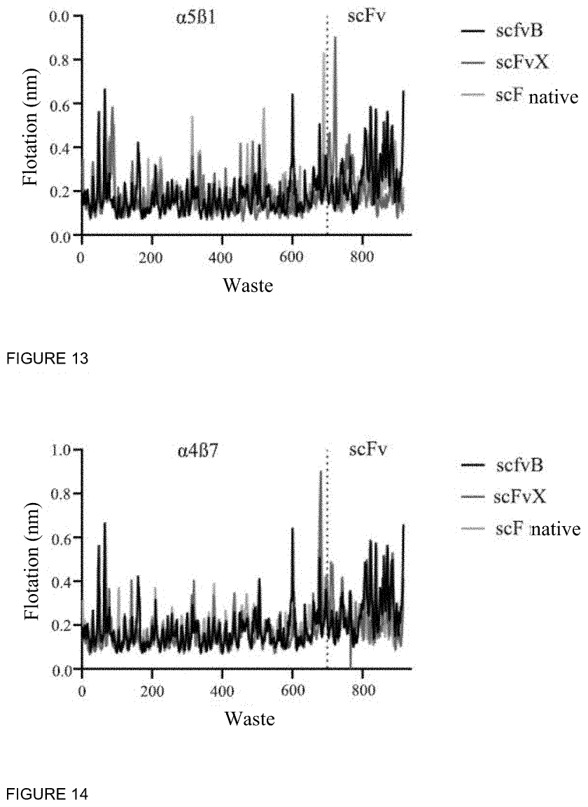

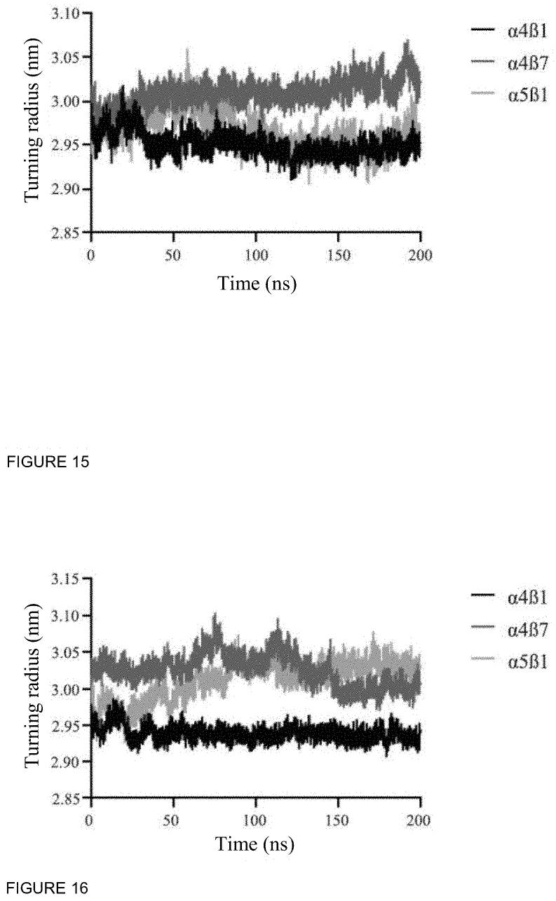

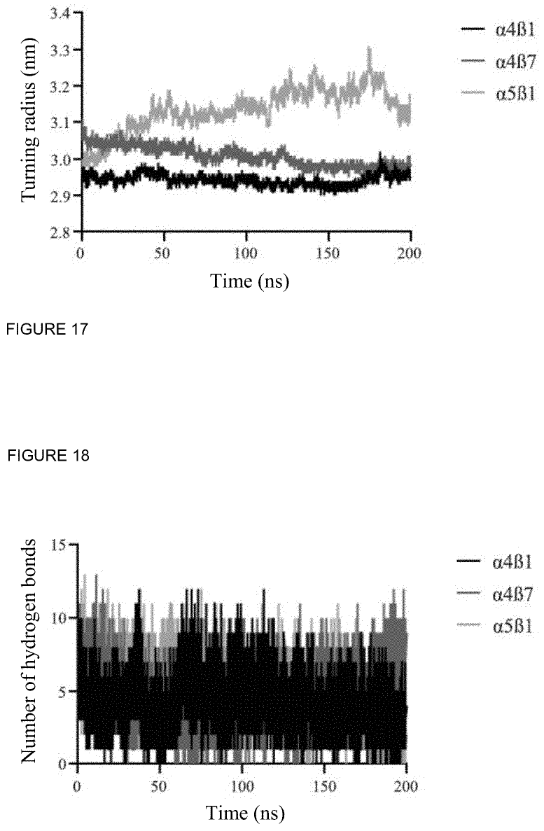

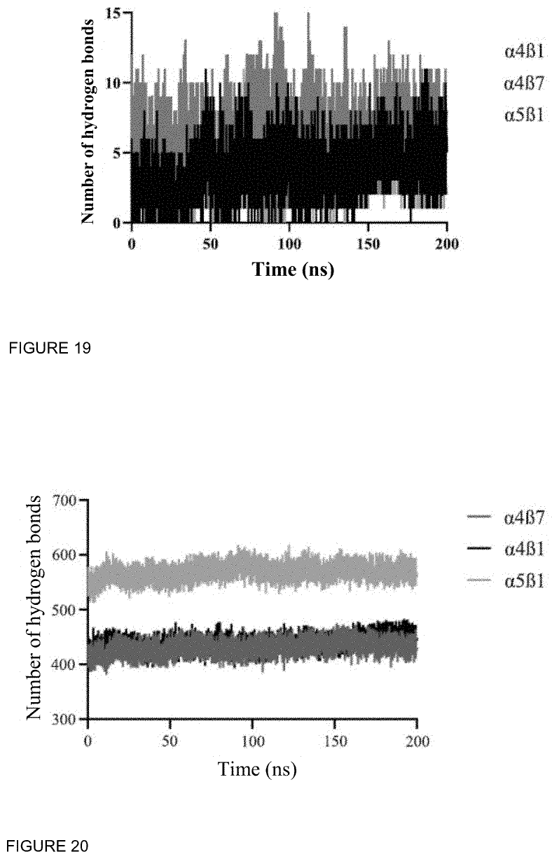

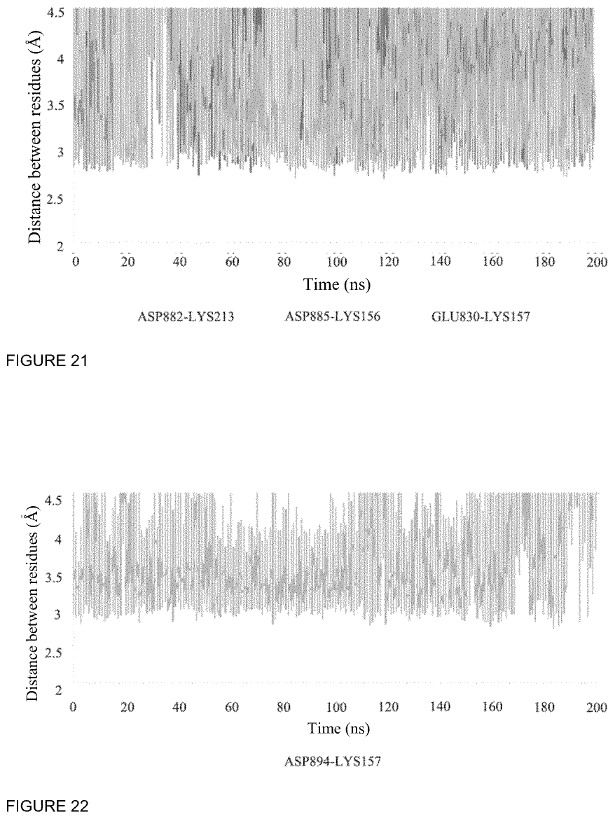

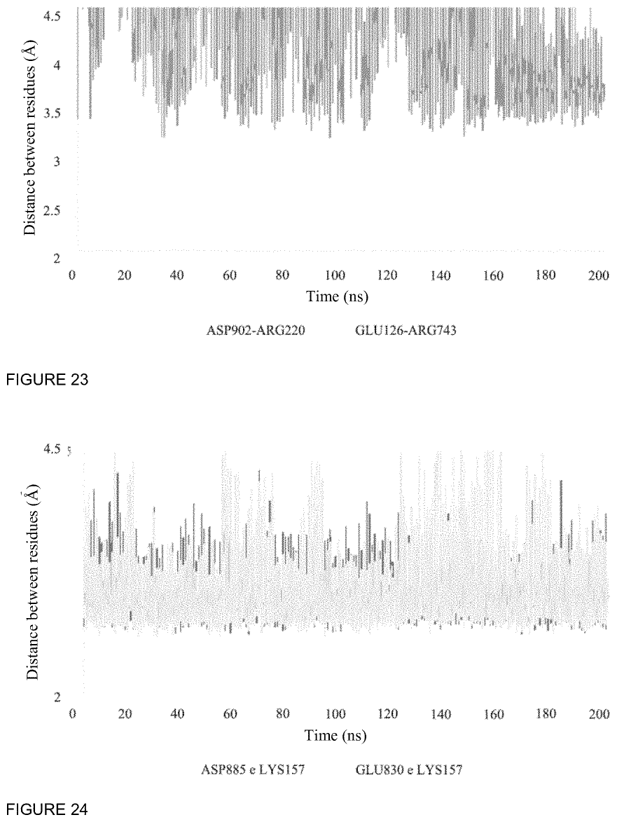

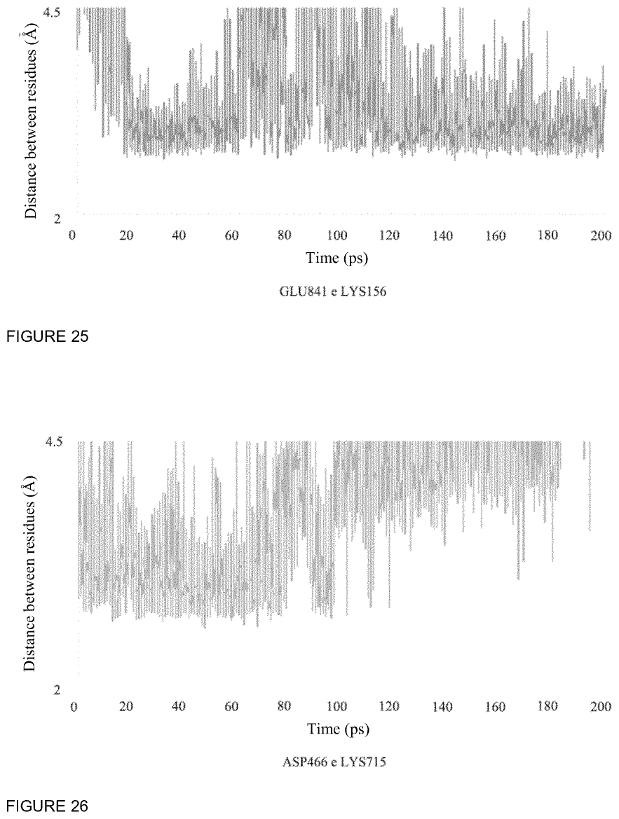

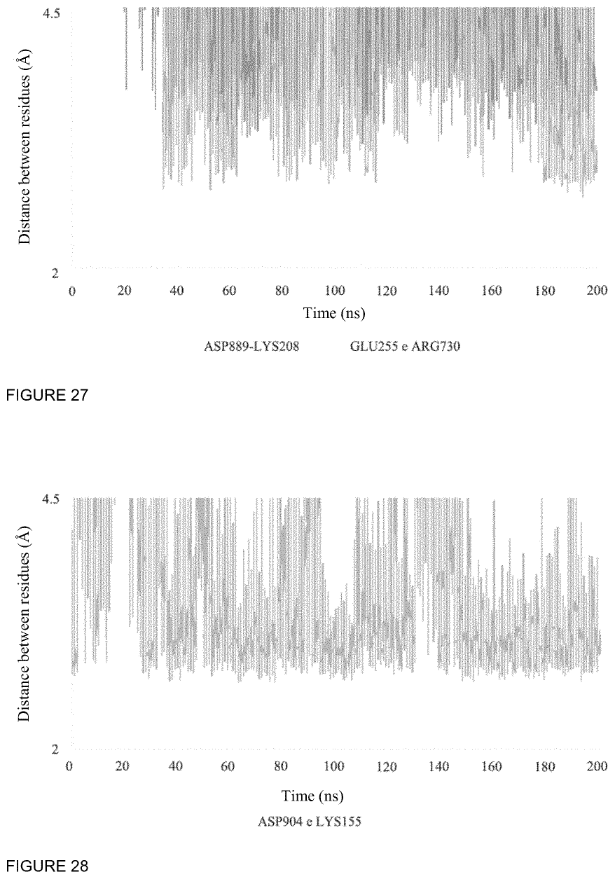

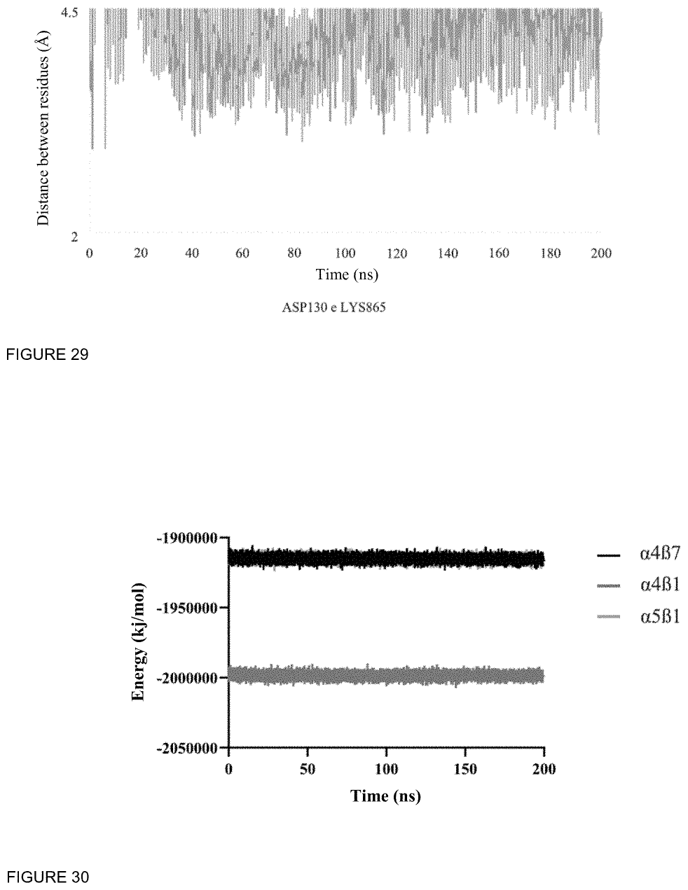

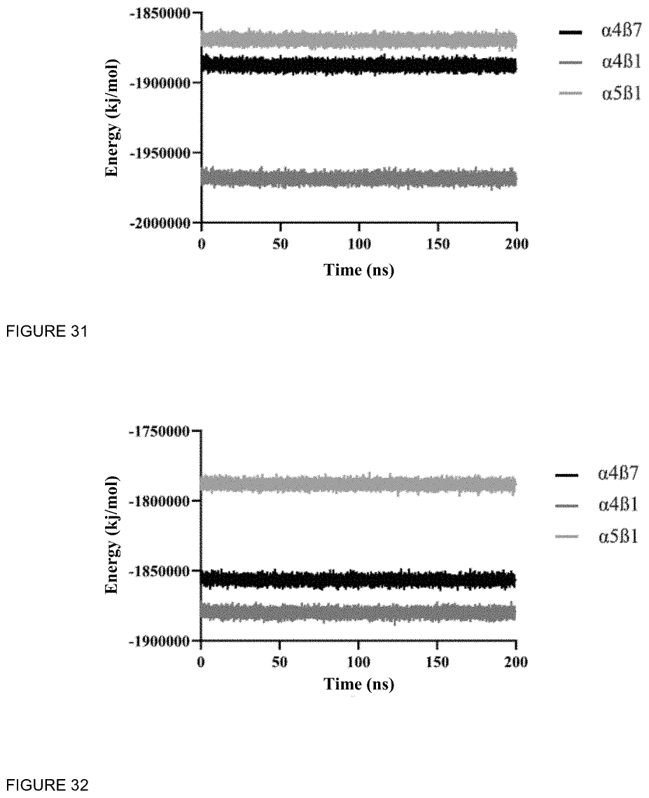

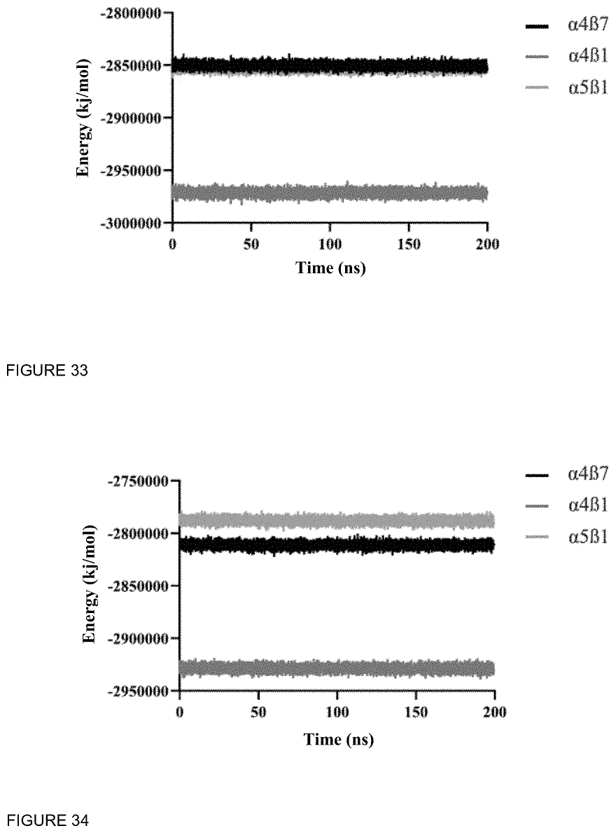

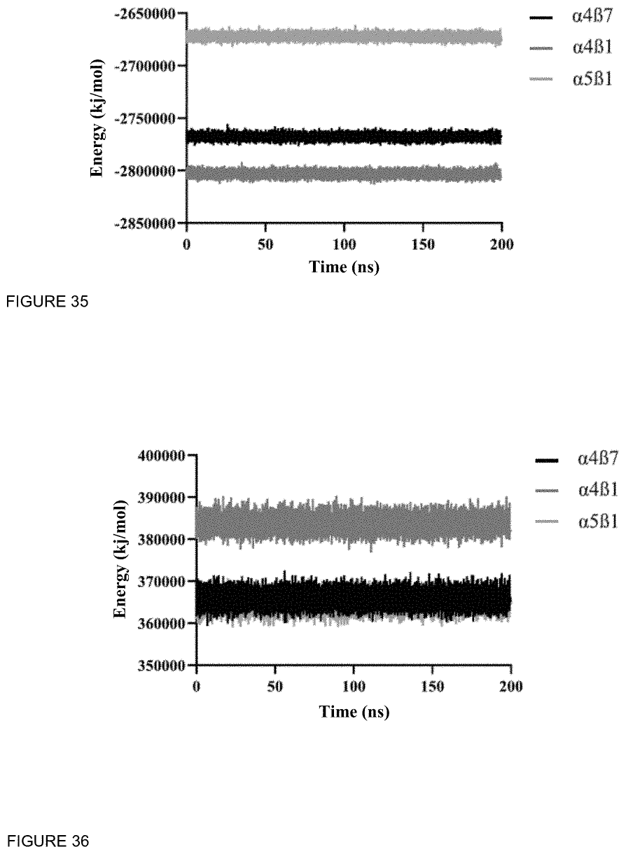

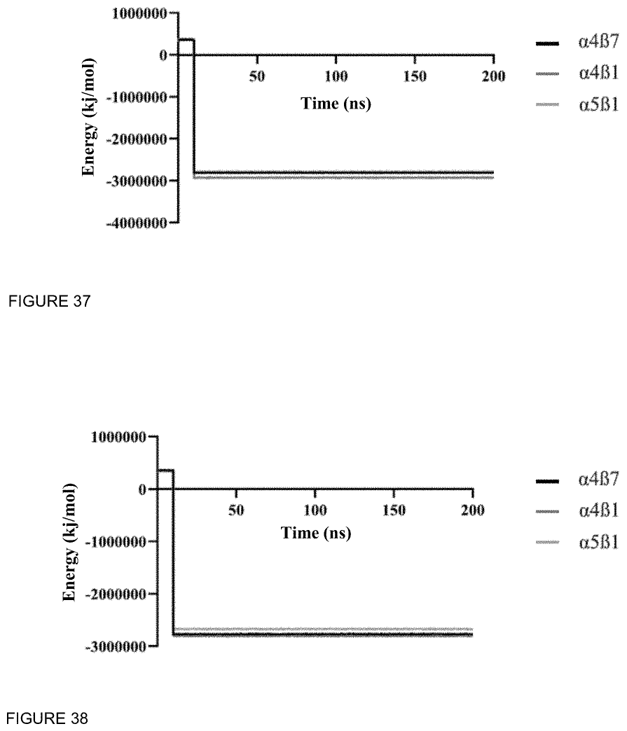

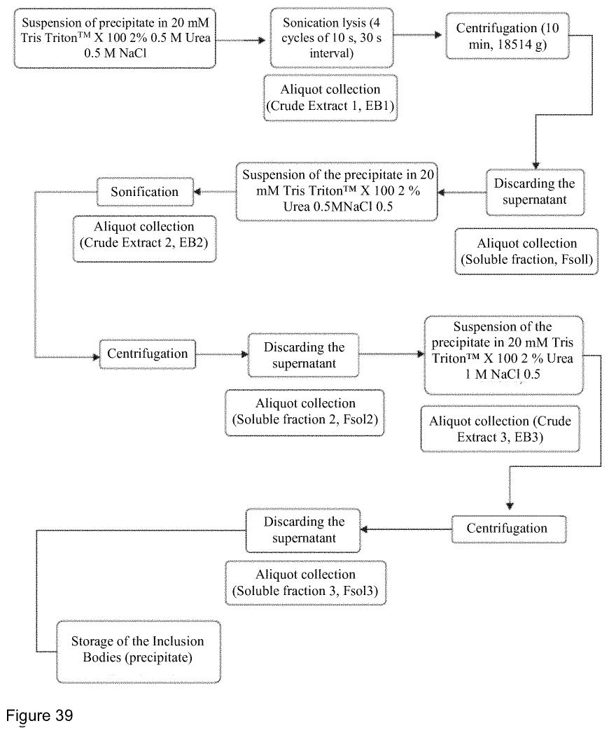

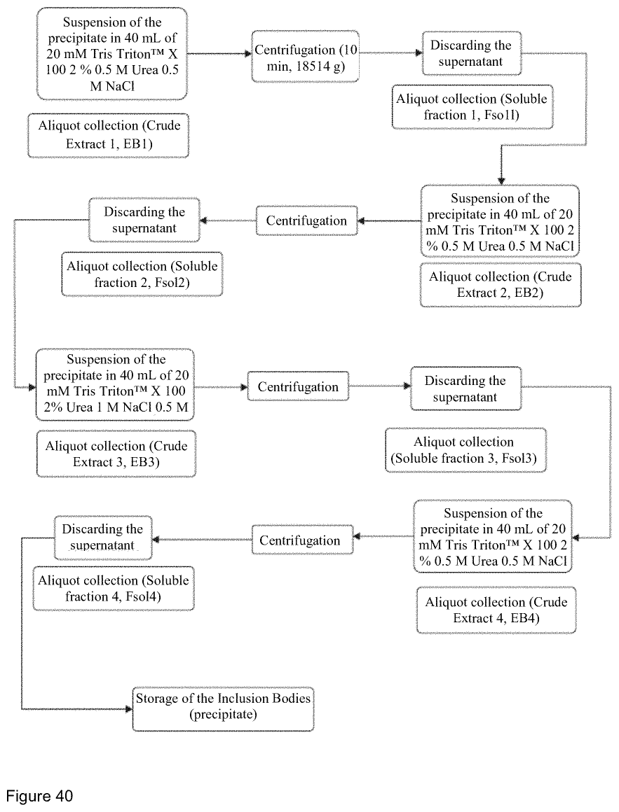

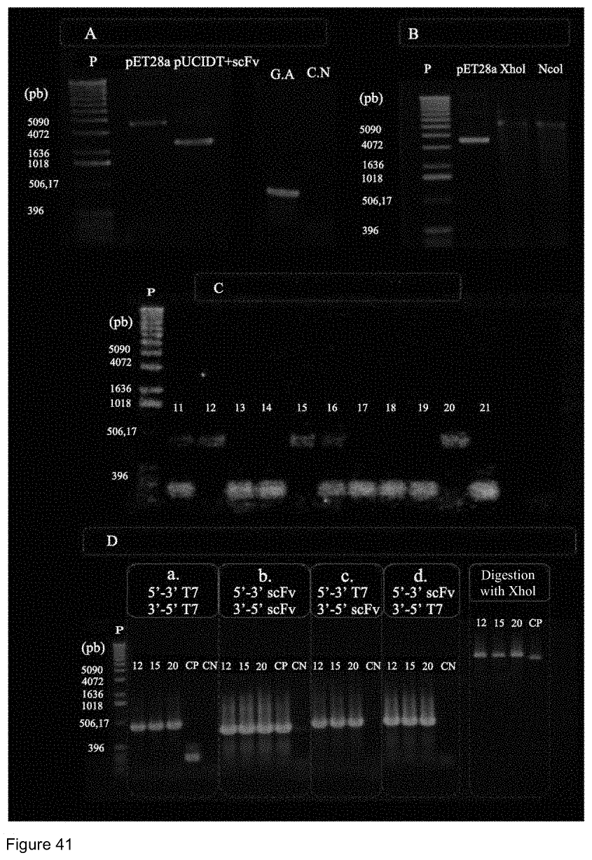

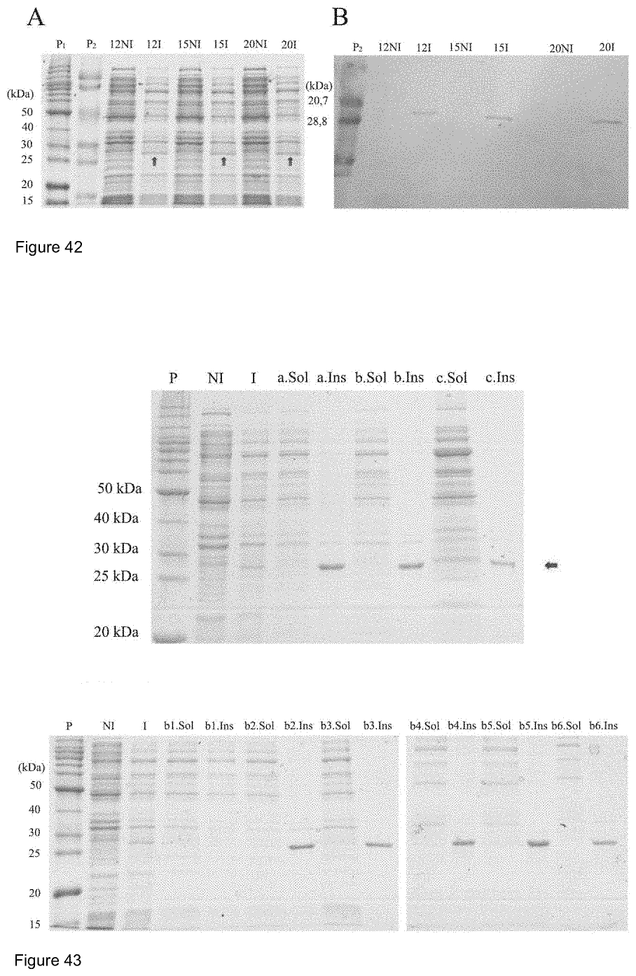

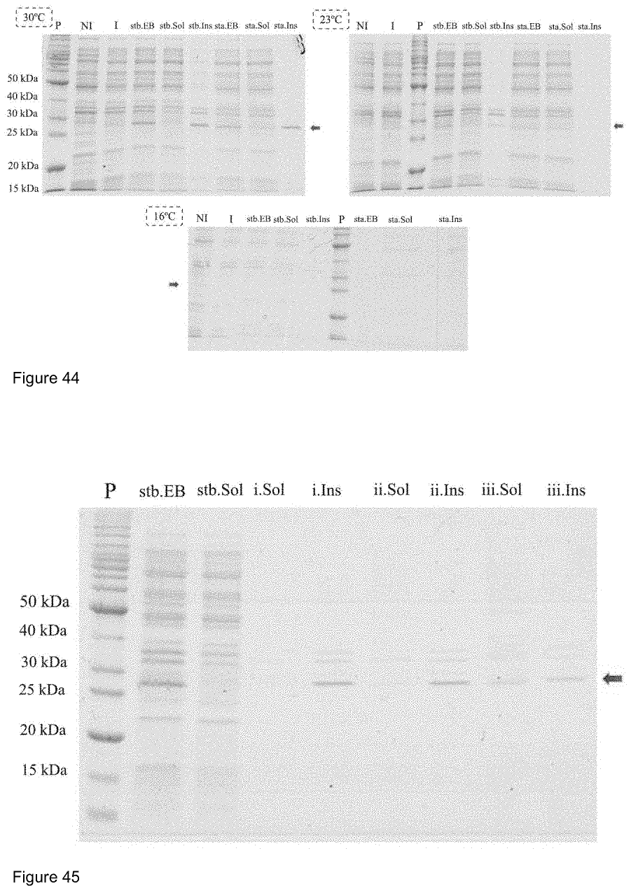

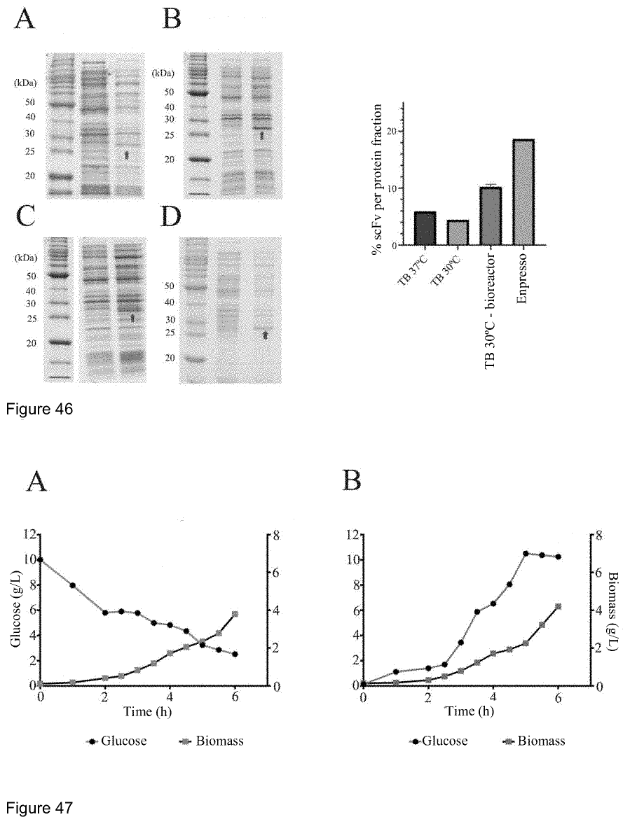

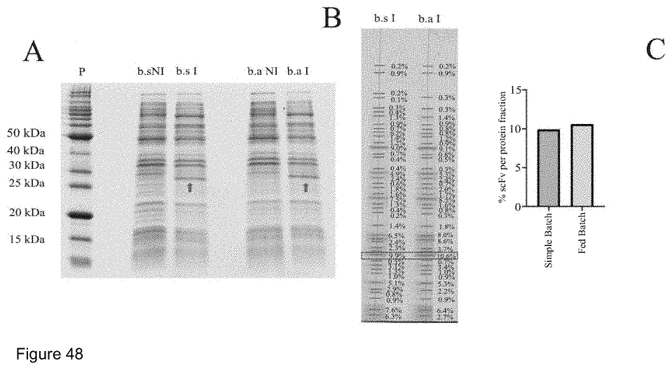

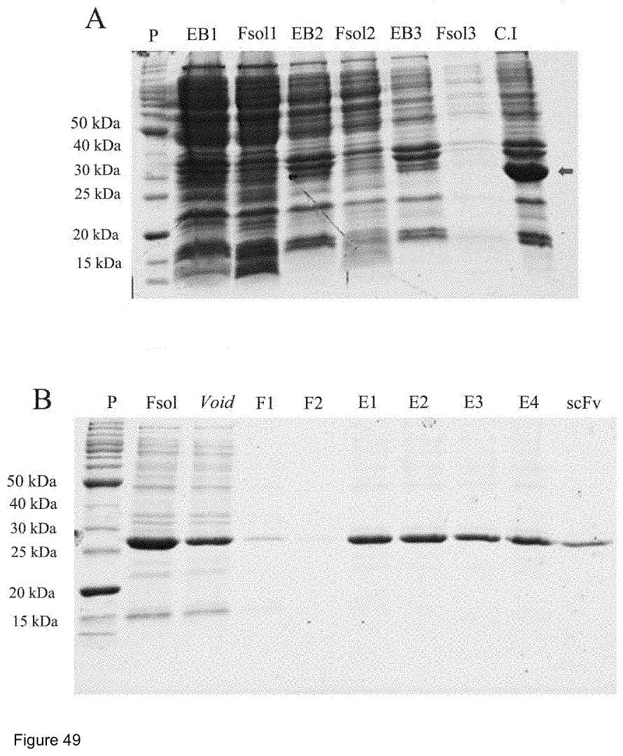

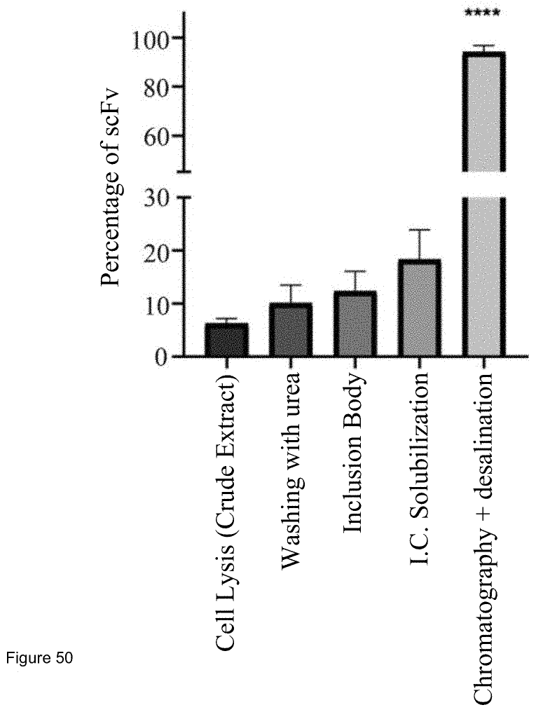

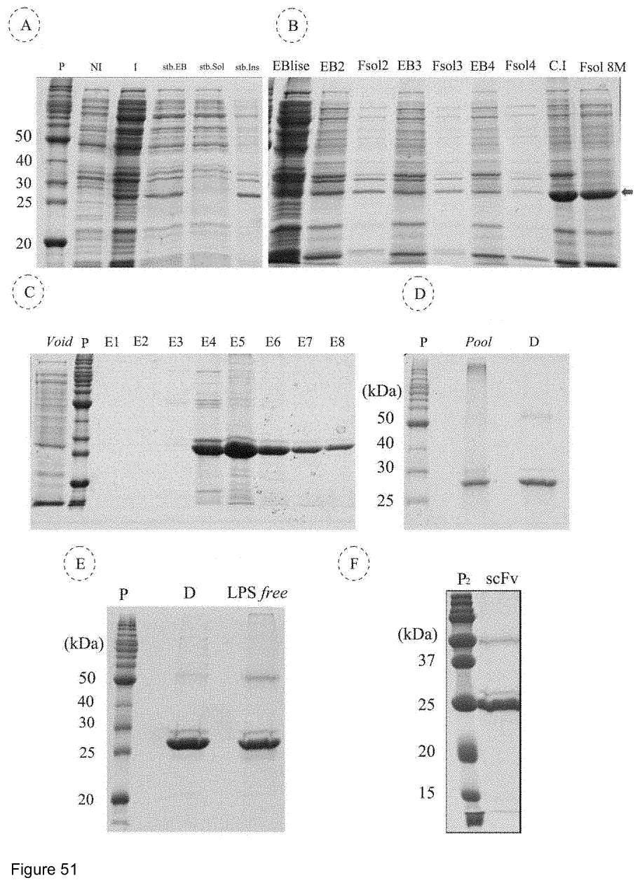

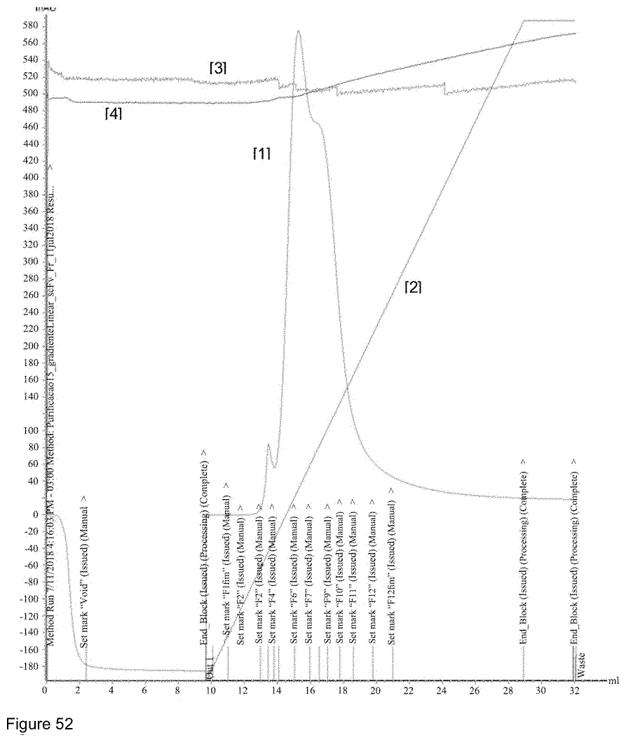

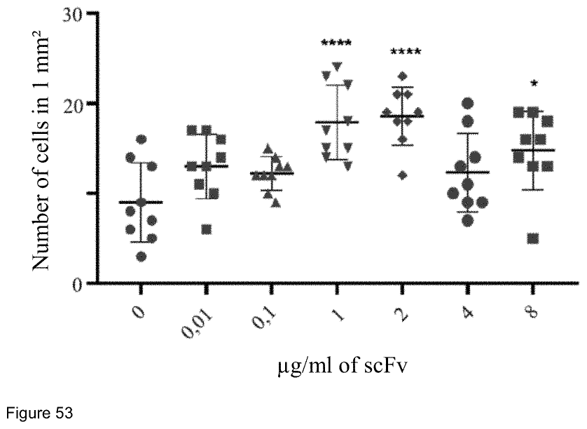

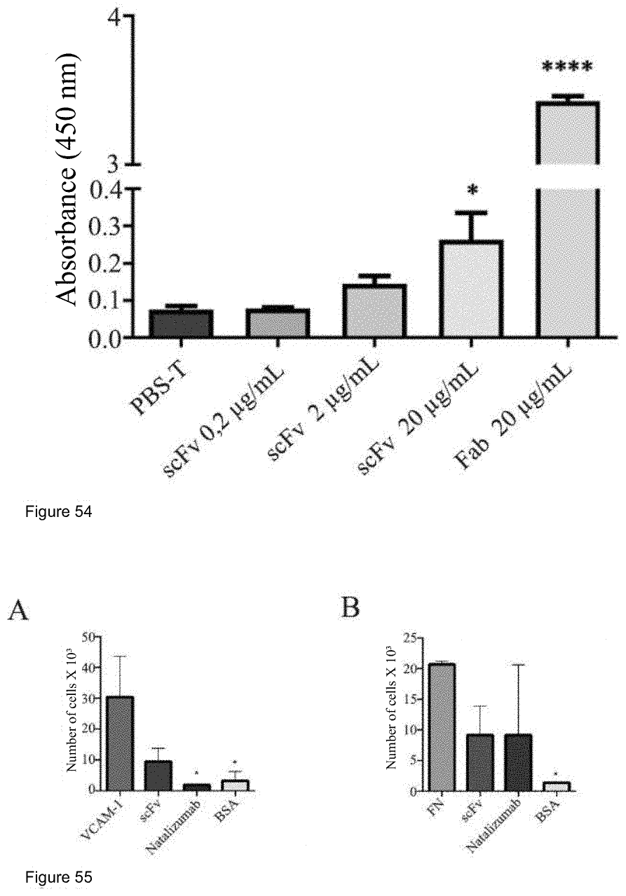

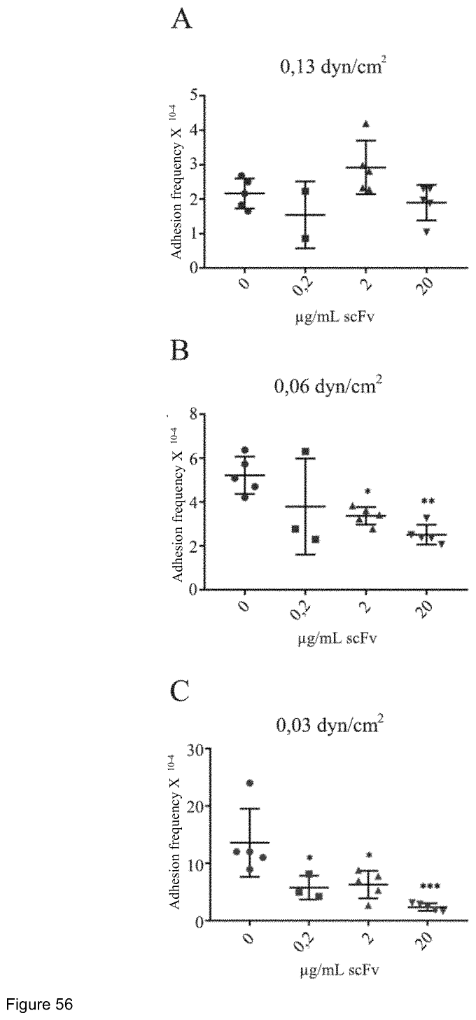

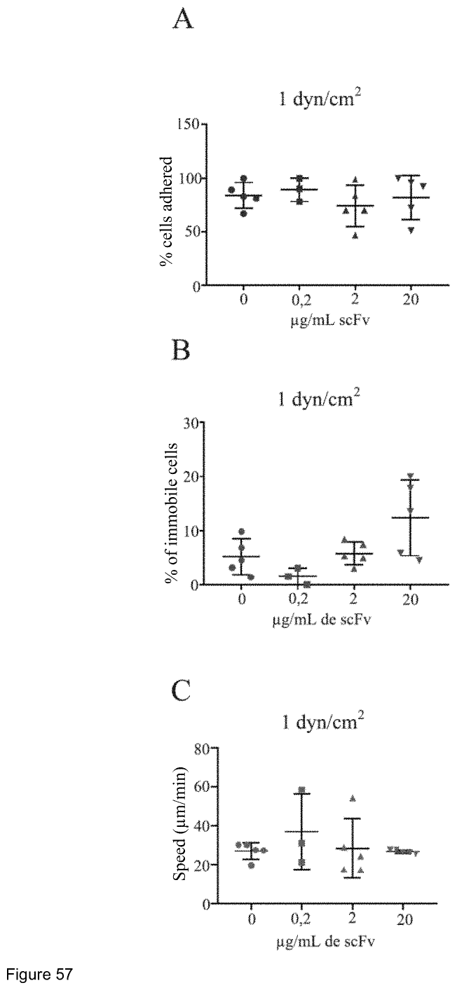

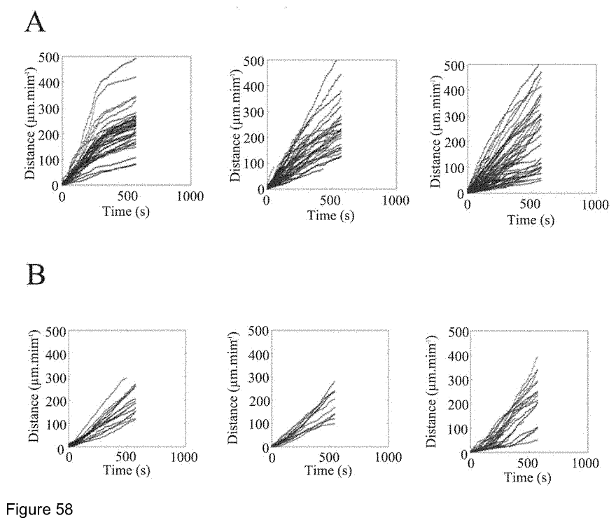

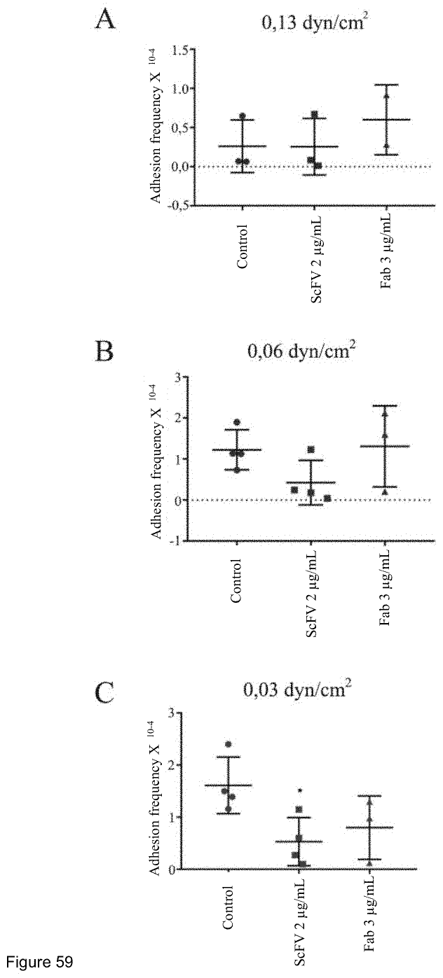

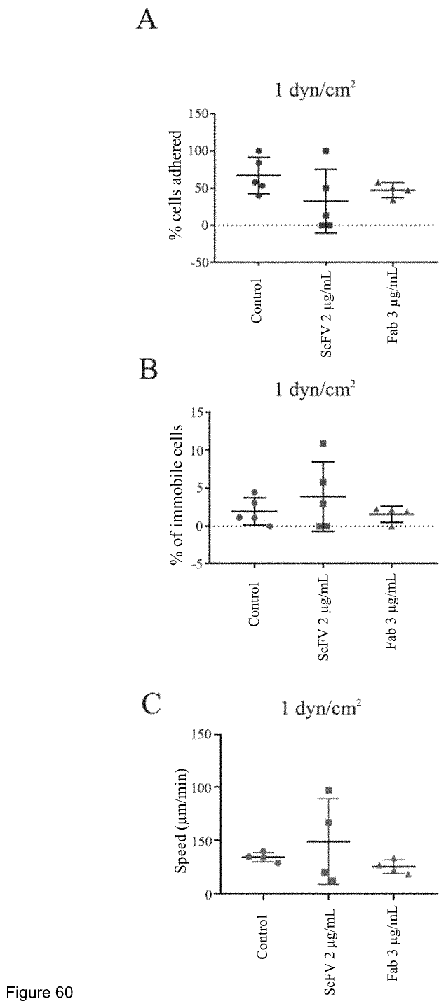

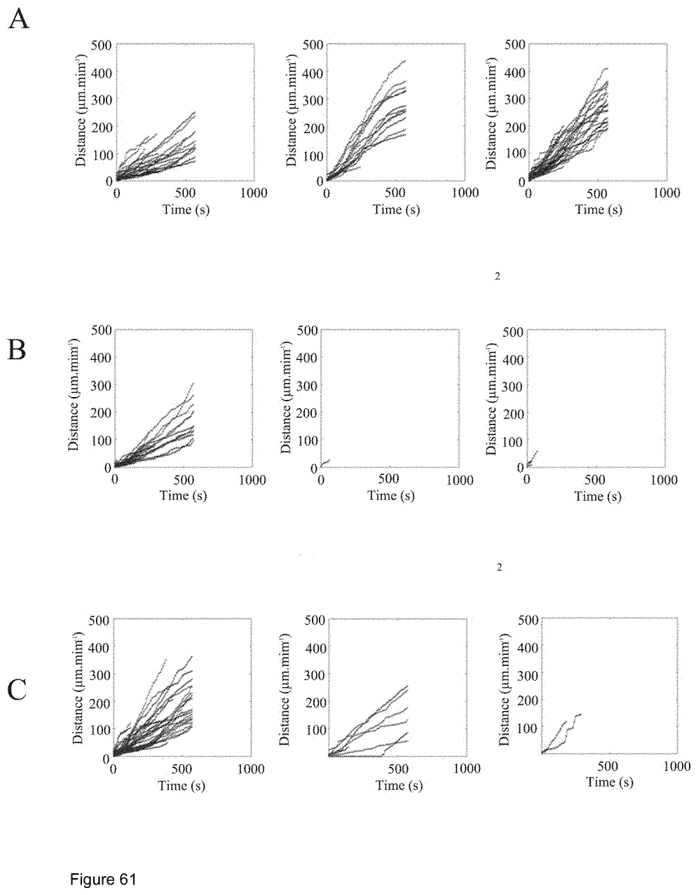

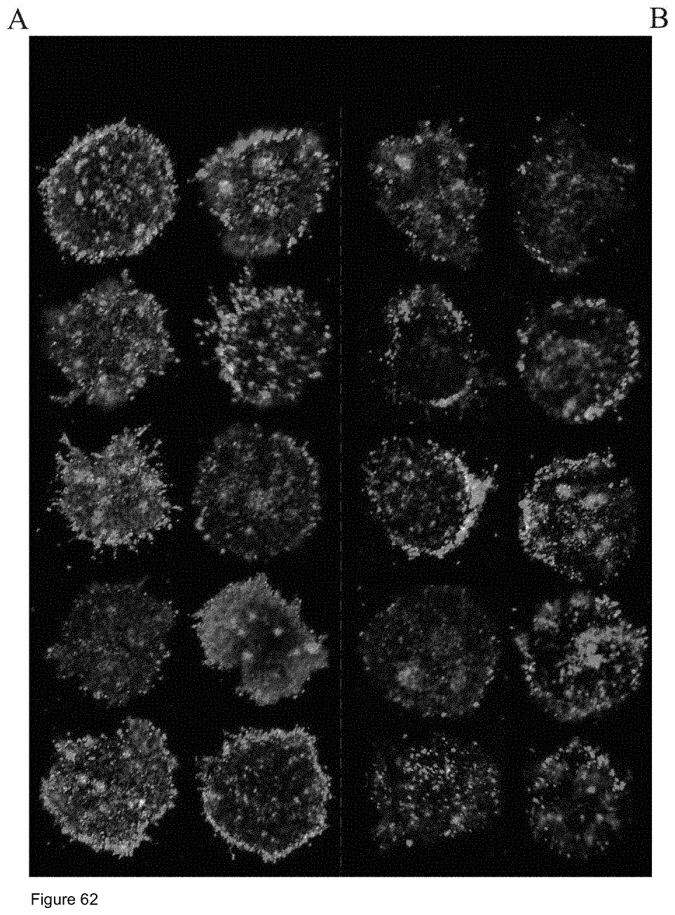

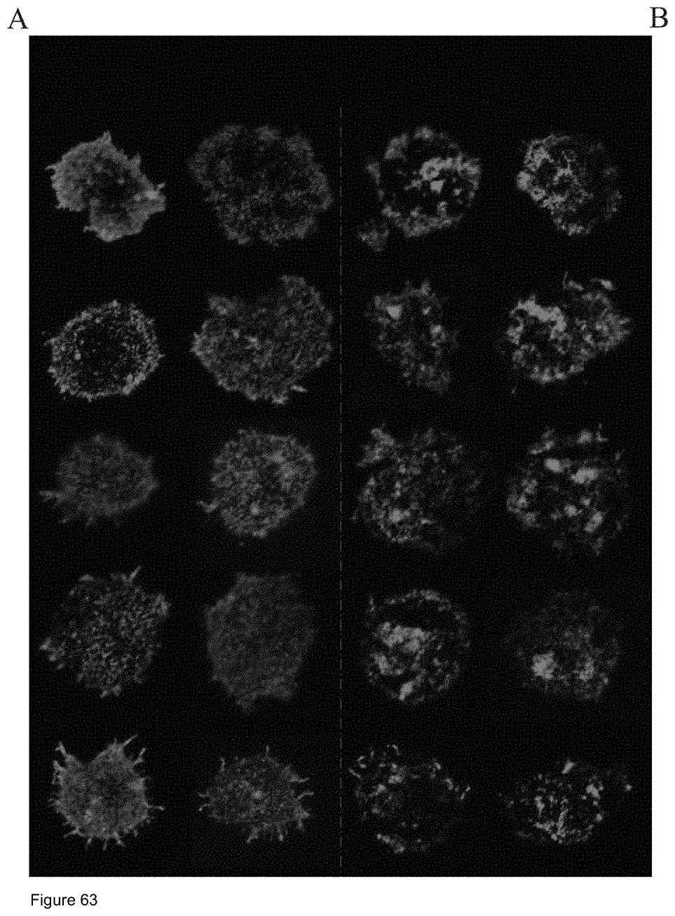

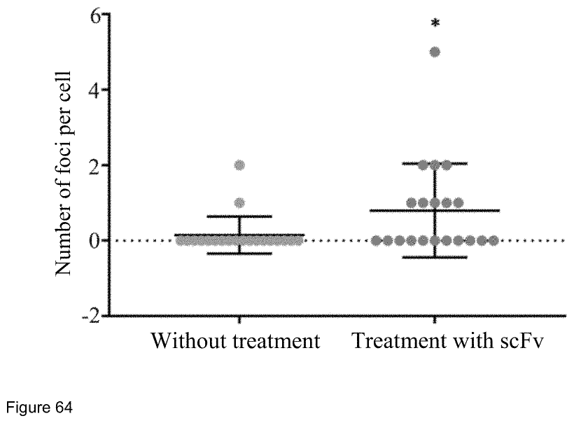

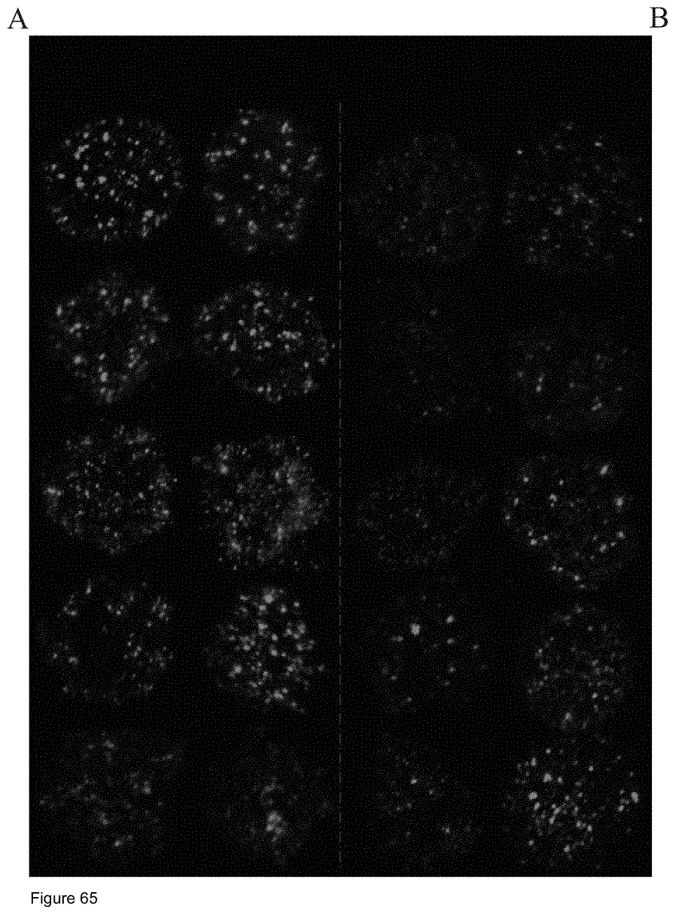

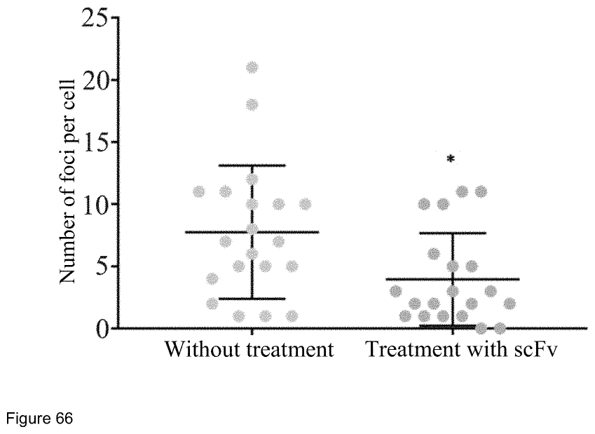

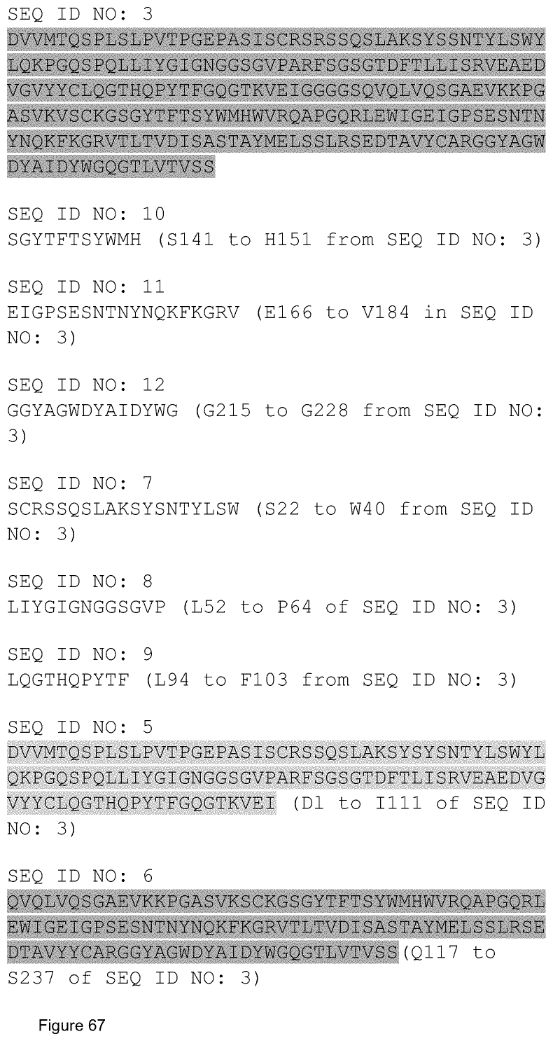

It was reported by Gao et al. (Circ Res. 2010; 107: 1445-1453) that myocardial IRI was induced using an artificial ventilation-free method. 8-10 week-old C57BL/6 mice were selected and divided into an experimental group and a control group; and the experimental group (N38-Fc) was injected with N38-Fc (8 mg/kg) and the control group (Vehicle) was injected with a same volume of PBS. The first injection was conducted 5 min before surgery, and then injection was conducted every 6 h. The mice were allowed to inhale 3% isoflurane for anaesthetization and then to inhale 1.5% to 2% isoflurane for anaesthetization maintenance; the mice were placed at a supine position, the skin on the left chest was cut, a chest muscle was simply separated, and then the chest cavity was rapidly exposed by left fourth intercostal thoracotomy; the pericardium was opened to expose the mice, and the left anterior descending (LAD) coronary artery 2 mm to 3 mm from the start was ligated with a 7-0 silk suture through a slipknot; the success ligation was confirmed by the simultaneous occurrence of anterior wall whitening of the left ventricle and ST segment elevation of electrocardiogram (ECG); then the heart was quickly returned to the chest cavity, the air was manually evacuated, and the chest cavity was closed with a 4-0 suture; one inner end of a slipknot suture was cut as short as possible, and the other end was about 0.8 cm long and remained outside the chest cavity; and the anaesthetization was then stopped, and the animal was allowed to recover. After 30 min of ischaemia, the mice were anesthetized once again, and the slipknot was loosened by smoothly pulling a long end of the suture until a complete release was achieved, at which point myocardial reperfusion was started; and 24 h after ischemia-reperfusion, the cardiac function and ventricular structure were determined through echocardiography (VisualSonics VeVo 2100 imaging system) by assessing EF, left ventricular FS, left ventricular anterolateral wall (LVAW) thickness, left ventricular posterior wall (LVPW), left ventricular volume, and left ventricular mass. The groups had a similar mortality of about 20%. After 24 h of cardiac reperfusion, the LAD was occluded once again in the previous position, and a 2% Evans blue dye (Sigma, Darmstadt, Germany) was injected into the cardiac chamber through the ascending aorta; the mice were then euthanized, the heart was collected, rinsed with PBS, frozen at −80° C. for 30 min, and cut crosswise below the ligation line into 5 sections; the sections were incubated with 1% 2,3,5-triphenyltetrazolium chloride (TTC, Amresco, America) for 10 min in a dark room at 37° C. and then fixed with formalin for 2 h; images were acquired with a stereomicroscope (Zeiss, Germany) (results were shown in FIG. 12A); and the Image-Pro Plus 6.0 software (Media Cyberneics) was used to measure and calculate an ischemic area, an infarct tissue, and a left ventricular area.

FIGS. 12A-12F show an effect of N38-Fc intervention on IS in AAR, and it can be seen that the N38-Fc intervention significantly reduces an IS percentage in the AAR, where the groups have a similar AAR (as shown in FIGS. 12A-12C). The echocardiography results show that, compared with the control, the MI/R-induced cardiac systolic dysfunction such as left ventricular EF (EF %) and FS (FS %) (as shown in FIGS. 12D-12F) was significantly improved in N38-Fc-treated mice. These results suggest that the FXII nanobody intervention has a protective effect for myocardial IRI.

The above examples are merely few examples of the present application, and do not limit the present application in any form. Although the present application is disclosed as above with preferred examples, the present application is not limited thereto. Some changes or modifications made by any technical personnel familiar with the profession using the technical content disclosed above without departing from the scope of the technical solutions of the present application are equivalent to equivalent implementation cases and fall within the scope of the technical solutions.

What is claimed is:

1. An anti-factor XII (FXII) nanobody or an antigen-binding fragment thereof, wherein the anti-FXII nanobody or the antigen-binding fragment thereof binds to a FXII through a binding epitope of the FXII to block an activation of the FXII, and the binding epitope of the FXII comprises a conformational epitope.

2. The anti-FXII nanobody or the antigen-binding fragment thereof according to claim 1, wherein a binding epitope of the anti-FXII nanobody or the antigen-binding fragment thereof to the FXII is a conformational epitope and is configured to bind to a fibronectin type II domain and a kringle domain of the FXII.

3. The anti-FXII nanobody or the antigen-binding fragment thereof according to claim 1, wherein the anti-FXII nanobody or the antigen-binding fragment thereof has almost no ability to block an activity of a FXIIa.

4. The anti-FXII nanobody or the antigen-binding fragment thereof according to claim 1, wherein when a molar ratio of the anti-FXII nanobody or the antigen-binding fragment thereof to the FXII is 1:(0.1-0.3), a blocking efficiency of the anti-FXII nanobody or the antigen-binding fragment thereof for the FXII is no less than 50%.

5. The anti-FXII nanobody or the antigen-binding fragment thereof according to claim 1, wherein the anti-FXII nanobody or the antigen-binding fragment thereof is derived from an alpaca.

6. The anti-FXII nanobody or the antigen-binding fragment thereof according to claim 1, wherein the anti-FXII nanobody or the antigen-binding fragment thereof is a recombinant antibody.

7. The anti-FXII nanobody or the antigen-binding fragment thereof according to claim 6, wherein the recombinant antibody comprises a nanobody fused with an immunoglobulin Fc fragment.

8. The anti-FXII nanobody or the antigen-binding fragment thereof according to claim 1, wherein based on a Kabat Database analysis,
n-1. the anti-FXII nanobody comprises a heavy chain CDR-H1 with a sequence shown in Sequence NO: 1, a heavy chain CDR-H2 with a sequence shown in Sequence NO: 2, and a heavy chain CDR-H3 with a sequence shown in Sequence NO: 3; or
n-2. the anti-FXII nanobody comprises a heavy chain CDR-H1 with a sequence shown in Sequence NO: 5, a heavy chain CDR-H2 with a sequence shown in Sequence NO: 6, and a heavy chain CDR-H3 with a sequence shown in Sequence NO: 7; or
n-3. the anti-FXII nanobody comprises a heavy chain CDR-H1 with a sequence shown in Sequence NO: 9, a heavy chain CDR-H2 with a sequence shown in Sequence NO: 10, and a heavy chain CDR-H3 with a sequence shown in Sequence NO: 11; or
n-4. the anti-FXII nanobody comprises a heavy chain CDR-H1 with a sequence shown in Sequence NO: 13, a heavy chain CDR-H2 with a sequence shown in Sequence NO: 14, and a heavy chain CDR-H3 with a sequence shown in Sequence NO: 15; or
n-5. the anti-FXII nanobody comprises a heavy chain CDR-H1 with a sequence shown in Sequence NO: 17, a heavy chain CDR-H2 with a sequence shown in Sequence NO: 18, and a heavy chain CDR-H3 with a sequence shown in Sequence NO: 19; or
n-6. the anti-FXII nanobody comprises a heavy chain CDR-H1 with a sequence shown in Sequence NO: 21, a heavy chain CDR-H2 with a sequence shown in Sequence NO: 22, and a heavy chain CDR-H3 with a sequence shown in Sequence NO: 23; or
n-7. the anti-FXII nanobody comprises a heavy chain CDR-H1 with a sequence shown in Sequence NO: 25, a heavy chain CDR-H2 with a sequence shown in Sequence NO: 26, and a heavy chain CDR-H3 with a sequence shown in Sequence NO: 27; or
n-8. the anti-FXII nanobody comprises a heavy chain CDR-H1 with a sequence shown in Sequence NO: 29, a heavy chain CDR-H2 with a sequence shown in Sequence NO: 30, and a heavy chain CDR-H3 with a sequence shown in Sequence NO: 31; or
n-9. the anti-FXII nanobody comprises a heavy chain CDR-H1 with a sequence shown in Sequence NO: 33, a heavy chain CDR-H2 with a sequence shown in Sequence NO: 34, and a heavy chain CDR-H3 with a sequence shown in Sequence NO: 35; or
n-10. the anti-FXII nanobody comprises a heavy chain CDR-H1 with a sequence shown in Sequence NO: 37, a heavy chain CDR-H2 with a sequence shown in Sequence NO: 38, and a heavy chain CDR-H3 with a sequence shown in Sequence NO: 39; or
n-11. the anti-FXII nanobody comprises a heavy chain CDR-H1 with a sequence shown in Sequence NO: 41, a heavy chain CDR-H2 with a sequence shown in Sequence NO: 42, and a heavy chain CDR-H3 with a sequence shown in Sequence NO: 43; or
n-12. the anti-FXII nanobody comprises a heavy chain CDR-H1 with a sequence shown in Sequence NO: 45, a heavy chain CDR-H2 with a sequence shown in Sequence NO: 46, and a heavy chain CDR-H3 with a sequence shown in Sequence NO: 47; or
n-13. the anti-FXII nanobody comprises a heavy chain CDR-H1 with a sequence shown in Sequence NO: 49, a heavy chain CDR-H2 with a sequence shown in Sequence NO: 50, and a heavy chain CDR-H3 with a sequence shown in Sequence NO: 51; or
n-14. the anti-FXII nanobody comprises a heavy chain CDR-H1 with a sequence shown in Sequence NO: 53, a heavy chain CDR-H2 with a sequence shown in Sequence NO: 54, and a heavy chain CDR-H3 with a sequence shown in Sequence NO: 55; or
n-15. the anti-FXII nanobody comprises a heavy chain CDR-H1 with a sequence shown in Sequence NO: 57, a heavy chain CDR-H2 with a sequence shown in Sequence NO: 58, and a heavy chain CDR-H3 with a sequence shown in Sequence NO: 59; or
n-16. the anti-FXII nanobody comprises a heavy chain CDR-H1 with a sequence shown in Sequence NO: 61, a heavy chain CDR-H2 with a sequence shown in Sequence NO: 62, and a heavy chain CDR-H3 with a sequence shown in Sequence NO: 63; or
n-17. the anti-FXII nanobody comprises a heavy chain CDR-H1 with a sequence shown in Sequence NO: 65, a heavy chain CDR-H2 with a sequence shown in Sequence NO: 66, and a heavy chain CDR-H3 with a sequence shown in Sequence NO: 67; and
based on an IMGT Database analysis,
n-1. the anti-FXII nanobody comprises a heavy chain CDR-H1 with a sequence shown in Sequence NO: 69, a heavy chain CDR-H2 with a sequence shown in Sequence NO: 70, and a heavy chain CDR-H3 with a sequence shown in Sequence NO: 71; or
n-2. the anti-FXII nanobody comprises a heavy chain CDR-H1 with a sequence shown in Sequence NO: 73, a heavy chain CDR-H2 with a sequence shown in Sequence NO: 74, and a heavy chain CDR-H3 with a sequence shown in Sequence NO: 75; or
n-3. the anti-FXII nanobody comprises a heavy chain CDR-H1 with a sequence shown in Sequence NO: 77, a heavy chain CDR-H2 with a sequence shown in Sequence NO: 78, and a heavy chain CDR-H3 with a sequence shown in Sequence NO: 79; or
n-4. the anti-FXII nanobody comprises a heavy chain CDR-H1 with a sequence shown in Sequence NO: 81, a heavy chain CDR-H2 with a sequence shown in Sequence NO: 82, and a heavy chain CDR-H3 with a sequence shown in Sequence NO: 83; or
n-5. the anti-FXII nanobody comprises a heavy chain CDR-H1 with a sequence shown in Sequence NO: 85, a heavy chain CDR-H2 with a sequence shown in Sequence NO: 86, and a heavy chain CDR-H3 with a sequence shown in Sequence NO: 87; or
n-6. the anti-FXII nanobody comprises a heavy chain CDR-H1 with a sequence shown in Sequence NO: 89, a heavy chain CDR-H2 with a sequence shown in Sequence NO: 90, and a heavy chain CDR-H3 with a sequence shown in Sequence NO: 91; or
n-7. the anti-FXII nanobody comprises a heavy chain CDR-H1 with a sequence shown in Sequence NO: 93, a heavy chain CDR-H2 with a sequence shown in Sequence NO: 94, and a heavy chain CDR-H3 with a sequence shown in Sequence NO: 95; or
n-8. the anti-FXII nanobody comprises a heavy chain CDR-H1 with a sequence shown in Sequence NO: 97, a heavy chain CDR-H2 with a sequence shown in Sequence NO: 98, and a heavy chain CDR-H3 with a sequence shown in Sequence NO: 99; or
n-9. the anti-FXII nanobody comprises a heavy chain CDR-H1 with a sequence shown in Sequence NO: 101, a heavy chain CDR-H2 with a sequence shown in Sequence NO: 102, and a heavy chain CDR-H3 with a sequence shown in Sequence NO: 103; or
n-10. the anti-FXII nanobody comprises a heavy chain CDR-H1 with a sequence shown in Sequence NO: 105, a heavy chain CDR-H2 with a sequence shown in Sequence NO: 106, and a heavy chain CDR-H3 with a sequence shown in Sequence NO: 107; or
n-11. the anti-FXII nanobody comprises a heavy chain CDR-H1 with a sequence shown in Sequence NO: 109, a heavy chain CDR-H2 with a sequence shown in Sequence NO: 110, and a heavy chain CDR-H3 with a sequence shown in Sequence NO: 111; or
n-12. the anti-FXII nanobody comprises a heavy chain CDR-H1 with a sequence shown in Sequence NO: 113, a heavy chain CDR-H2 with a sequence shown in Sequence NO: 114, and a heavy chain CDR-H3 with a sequence shown in Sequence NO: 115; or
n-13. the anti-FXII nanobody comprises a heavy chain CDR-H1 with a sequence shown in Sequence NO: 117, a heavy chain CDR-H2 with a sequence shown in Sequence NO: 118, and a heavy chain CDR-H3 with a sequence shown in Sequence NO: 119; or
n-14. the anti-FXII nanobody comprises a heavy chain CDR-H1 with a sequence shown in Sequence NO: 121, a heavy chain CDR-H2 with a sequence shown in Sequence NO: 122, and a heavy chain CDR-H3 with a sequence shown in Sequence NO: 123; or
n-15. the anti-FXII nanobody comprises a heavy chain CDR-H1 with a sequence shown in Sequence NO: 125, a heavy chain CDR-H2 with a sequence shown in Sequence NO: 126, and a heavy chain CDR-H3 with a sequence shown in Sequence NO: 127; or
n-16. the anti-FXII nanobody comprises a heavy chain CDR-H1 with a sequence shown in Sequence NO: 129, a heavy chain CDR-H2 with a sequence shown in Sequence NO: 130, and a heavy chain CDR-H3 with a sequence shown in Sequence NO: 131; or
n-17. the anti-FXII nanobody comprises a heavy chain CDR-H1 with a sequence shown in Sequence NO: 133, a heavy chain CDR-H2 with a sequence shown in Sequence NO: 134, and a heavy chain CDR-H3 with a sequence shown in Sequence NO: 135.

9. The anti-FXII nanobody or the antigen-binding fragment thereof according to claim 8, wherein based on the Kabat Database analysis,
n-101. the anti-FXII nanobody comprises a heavy chain variable region with a sequence shown in SEQ ID NO: 4; or
n-102. the anti-FXII nanobody comprises a heavy chain variable region with a sequence shown in SEQ ID NO: 8; or
n-103. the anti-FXII nanobody comprises a heavy chain variable region with a sequence shown in SEQ ID NO: 12; or
n-104. the anti-FXII nanobody comprises a heavy chain variable region with a sequence shown in SEQ ID NO: 16; or
n-105. the anti-FXII nanobody comprises a heavy chain variable region with a sequence shown in SEQ ID NO: 20; or
n-106. the anti-FXII nanobody comprises a heavy chain variable region with a sequence shown in SEQ ID NO: 24; or
n-107. the anti-FXII nanobody comprises a heavy chain variable region with a sequence shown in SEQ ID NO: 28; or
n-108. the anti-FXII nanobody comprises a heavy chain variable region with a sequence shown in SEQ ID NO: 32; or
n-109. the anti-FXII nanobody comprises a heavy chain variable region with a sequence shown in SEQ ID NO: 36; or
n-110. the anti-FXII nanobody comprises a heavy chain variable region with a sequence shown in SEQ ID NO: 40; or
n-111. the anti-FXII nanobody comprises a heavy chain variable region with a sequence shown in SEQ ID NO: 44; or
n-112. the anti-FXII nanobody comprises a heavy chain variable region with a sequence shown in SEQ ID NO: 48; or
n-113. the anti-FXII nanobody comprises a heavy chain variable region with a sequence shown in SEQ ID NO: 52; or
n-114. the anti-FXII nanobody comprises a heavy chain variable region with a sequence shown in SEQ ID NO: 56; or
n-115. the anti-FXII nanobody comprises a heavy chain variable region with a sequence shown in SEQ ID NO: 60; or
n-116. the anti-FXII nanobody comprises a heavy chain variable region with a sequence shown in SEQ ID NO: 64; or
n-117. the anti-FXII nanobody comprises a heavy chain variable region with a sequence shown in SEQ ID NO: 68; and
based on the IMGT Database analysis,
n-101. the anti-FXII nanobody comprises a heavy chain variable region with a sequence shown in SEQ ID NO: 72; or
n-102. the anti-FXII nanobody comprises a heavy chain variable region with a sequence shown in SEQ ID NO: 76; or
n-103. the anti-FXII nanobody comprises a heavy chain variable region with a sequence shown in SEQ ID NO: 80; or
n-104. the anti-FXII nanobody comprises a heavy chain variable region with a sequence shown in SEQ ID NO: 84; or
n-105. the anti-FXII nanobody comprises a heavy chain variable region with a sequence shown in SEQ ID NO: 88; or
n-106. the anti-FXII nanobody comprises a heavy chain variable region with a sequence shown in SEQ ID NO: 92; or
n-107. the anti-FXII nanobody comprises a heavy chain variable region with a sequence shown in SEQ ID NO: 96; or
n-108. the anti-FXII nanobody comprises a heavy chain variable region with a sequence shown in SEQ ID NO: 100; or
n-109. the anti-FXII nanobody comprises a heavy chain variable region with a sequence shown in SEQ ID NO: 104; or
n-110. the anti-FXII nanobody comprises a heavy chain variable region with a sequence shown in SEQ ID NO: 108; or
n-111. the anti-FXII nanobody comprises a heavy chain variable region with a sequence shown in SEQ ID NO: 112; or
n-112. the anti-FXII nanobody comprises a heavy chain variable region with a sequence shown in SEQ ID NO: 116; or
n-113. the anti-FXII nanobody comprises a heavy chain variable region with a sequence shown in SEQ ID NO: 120; or
n-114. the anti-FXII nanobody comprises a heavy chain variable region with a sequence shown in SEQ ID NO: 124; or
n-115. the anti-FXII nanobody comprises a heavy chain variable region with a sequence shown in SEQ ID NO: 128; or
n-116. the anti-FXII nanobody comprises a heavy chain variable region with a sequence shown in SEQ ID NO: 132; or
n-117. the anti-FXII nanobody comprises a heavy chain variable region with a sequence shown in SEQ ID NO: 136.

10. A nucleic acid encoding the anti-FXII nanobody or the antigen-binding fragment thereof according to claim 1.

11. A vector comprising the nucleic acid according to claim 10, wherein the nucleic acid is effectively linked to an appropriate promoter sequence.

12. A prokaryotic cell, a cell line, a yeast cell, or a viral system comprising the vector according to claim 11.

13. (canceled)

14. A use of the antibody or the antigen-binding fragment thereof according to claim 1 in medicine.

15. A use of the anti-FXII nanobody or the antigen-binding fragment thereof according to claim 1 in a preparation of an antithrombotic drug.

16. An antithrombotic use of the anti-FXII nanobody or the antigen-binding fragment thereof according to claim 1 in an artificial medical device in contact with blood.

17. An antithrombotic use of the anti-FXII nanobody or the antigen-binding fragment thereof according to claim 1 in an extracorporeal membrane oxygenation (ECMO).

18. A use of the anti-FXII nanobody or the antigen-binding fragment thereof according to claim 1 in a preparation of an antivasculitis drug.

19. A use of the anti-FXII nanobody or the antigen-binding fragment thereof according to claim 1 in a preparation of an anti-cardiac ischemia-reperfusion injury (IRI) drug.

20. A pharmaceutical composition comprising the anti-FXII nanobody or the antigen-binding fragment thereof according to claim 1.
